(19) United States
(12) Patent Application Publication
(10) Pub. No.: US 2026/0079084 A1
(43) Pub. Date: Mar. 19, 2026

(54) COMPOUND, EXTRACELLULAR VESICLE STAINING AGENT, AND FLUORESCENT STAINING METHOD FOR EXTRACELLULAR VESICLES

(71) Applicant: National University Corporation Tokai National Higher Education and Research System, Nagoya-shi, Aichi (JP)

(72) Inventors: Hiroshi TAKEMORI, Gifu-shi, Gifu (JP); Kyoji FURUTA, Gifu-shi, Gifu (JP); YoKo MORITA, Gifu-shi, Gifu (JP); AKie HAMAMOTO, Gifu-shi, Gifu (JP)

(21) Appl. No.: 19/110,060

(22) PCT Filed: Aug. 8, 2023
(86) PCT No.: PCT/JP2023/028846

§ 371 Date: Mar. 7, 2025

(30) Foreign Application Priority Data
Sep. 7, 2022 (JP) 2022-141909

(51) Int. Cl. G01N 1/30; C07D 271/12; C07D 413/12

(52) U.S. Cl. CPC G01N 1/30; C07D 271/12; C07D 413/12; G01N 2001/302

(57) ABSTRACT

An object is to provide a novel compound expressed by the following Formula (1) that conjugates with an extracellular vesicle.

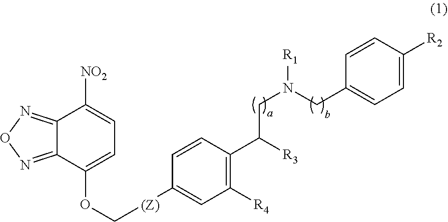

(in Formula (1), R₁ represents H, a C₁-C₆ alkyl group, a hydroxy group, an amino group, or a carboxy group, R₂ represents H, a C₁-C₁₈ alkyl group, a C₁-C₁₈ alkoxy group, NO₂ or N(CH₃)₂, and R₃ and R₄ each independently represent H or CH₃, where R₃ and R₄ may be bonded to each other to form a ring and, when R₃ and R₄ are bonded to each other to form a ring, R₃ and R₄ are CH₂, “a” represents 0 or 1, and “b” represents 1, 2, or 3, and Z represents a linker in which 0 to 12 chain molecules formed of elements selected from a group consisting of C, O, and N are bonded, and the chain molecules may include a cyclic structure or a branched chain.)

### TECHNICAL FIELD

The disclosure of the present application relates to a compound, an extracellular vesicle staining agent, and a method of fluorescent staining for extracellular vesicles.

### BACKGROUND ART

In recent years, roles of extracellular vesicles such as cellular vesicles, in particular, exosomes have attracted attention. Extracellular vesicles are not only present in body fluids such as blood or urine of humans but also contained in foods. These extracellular vesicles regulate functions of cells incorporated therein and thereby exert various effectiveness. It has been proposed that these extracellular vesicles act to enhance metabolism in skins.

In humans and mice, which are experimental animals, extracellular vesicles can be quantified by using markers on the surfaces thereof. On the other hand, for many other organism species without markers, systems for quantification and size measurement have been constructed by utilizing correlations exhibited between diameters of cellular vesicles and the Brownian motion, electrical resistances, or the like. However, there is a problem of inability of leading to accurate measurement, because the Brownian motion or electrical resistances are significantly affected by the compositions of solvents.

As another method for quantifying extracellular vesicles, a method of staining extracellular vesicles with a particular compound is also known (Patent Literature 1).

### CITATION LIST

### Patent Literature

Patent Literature 1: International Publication No. 2022/163446

### SUMMARY OF INVENTION

### Technical Problem

Through various experiments, however, the present inventors have newly found a problem that the compound disclosed in Patent Literature 1 can be used for detection of extracellular vesicles but not for size measurement such as liquid chromatography.

Accordingly, an object of the disclosure of the present application is to provide a novel compound that conjugates with extracellular vesicles and also enables size measurement such as liquid chromatography, an extracellular vesicle staining agent, and a method of fluorescent staining for extracellular vesicles.

### Solution to Problem

[1] A compound expressed by following Formula (1):

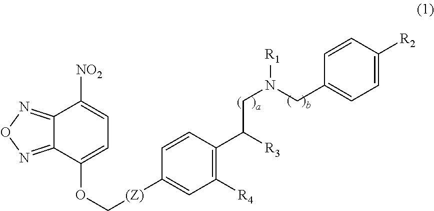

wherein in Formula (1), R₁ represents H, a C₁-C₆ alkyl group, a hydroxy group, an amino group, or a carboxy group, R₂ represents H, a C₁-C₁₈ alkyl group, a C₁-C₁₈ alkoxy group, NO₂ or N(CH₃)₂, and R₃ and R₄ each independently represent H or CH₃, where R₃ and R₄ may be bonded to each other to form a ring and, when R₃ and R₄ are bonded to each other to form a ring, R₃ and R₄ are CH₂, wherein “a” represents 0 or 1, and “b” represents 1, 2, or 3, and wherein Z represents a linker in which 0 to 12 chain molecules formed of elements selected from a group consisting of C, O, and N are bonded, and the chain molecules may include a cyclic structure or a branched chain.
[2] The compound according to [1] above, wherein the compound expressed by Formula (1) is a compound expressed by following Formula (2):

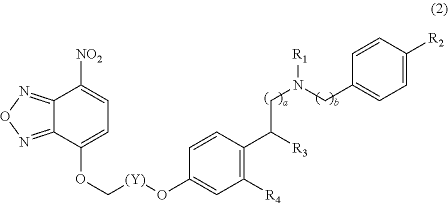

wherein in Formula (2), R₁ represents H or CH₃, R₂ represents H, CH₃, OCH₃, or N(CH₃)₂, R₃ and R₄ each independently represent H or CH₃, where R₃ and R₄ may be bonded to each other to form a ring and, when R₃ and R₄ are bonded to each other to form a ring, R₃ and R₄ are CH₂, wherein “a” represents 0 or 1, and “b” represents 1, 2, or 3, wherein Y represents a C₁-C₅ alkyl group, an ether chain represented by following Formula (2a), an ether chain including amido represented by following Formula (2b), or triazole represented by following Formula (2c),

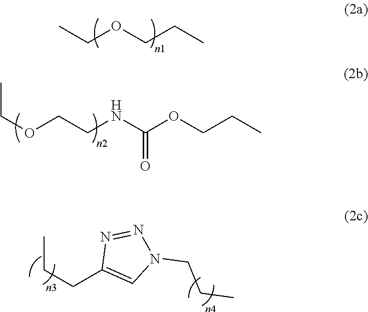

wherein in Formula (2a), n1 represents an integer of 1 to 3, wherein in Formula (2b), n2 represents an integer of 1 to 3, and wherein in Formula (2c), n3 represents an integer of 0 to 6, and n4 represents an integer of 0 to 3.
[3] The compound according to [1] above, wherein the compound expressed by Formula (1) is a compound expressed by following Formula (3):

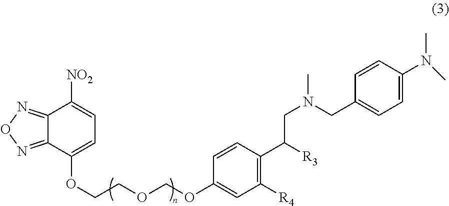

wherein in Formula (3), R₃ and R₄ each independently represent H or CH₃, where R₃ and R₄ may be bonded to each other to form a ring and, when R₃ and R₄ are bonded to each other to form a ring, R₃ and R₄ are CH₂, and wherein n represents an integer of 0 to 3.
[4] An extracellular vesicle staining agent comprising the compound according to [1] above.
[5] An extracellular vesicle staining agent comprising the compound according to [2]above.
[6] An extracellular vesicle staining agent comprising the compound according to [3] above.
[7] The extracellular vesicle staining agent according to [4] above further comprising another fluorescent compound.
[8] A method of fluorescent staining for extracellular vesicles, the method comprising:
a staining step of selecting any one of compounds according to [1] to [3] above or extracellular vesicle staining agents according to [4] to [6] above and staining extracellular vesicles by using the selected one; and
a detection step of detecting stained extracellular vesicles in a sample.
   [9] The method of fluorescent staining for extracellular vesicles according to [8] above further comprising an evaluation step of evaluating the sample after the detection step.
   [10] The method of fluorescent staining for extracellular vesicles according to [9] above,
wherein the sample includes cosmetics, and
wherein the method measures the extracellular vesicle incorporated in cells to evaluate the cosmetics.
   [11] The method of fluorescent staining for extracellular vesicles according to [9] above,
wherein the sample includes pharmaceutical products, and
wherein the method measures the extracellular vesicles incorporated in cells to evaluate the pharmaceutical product.
   [12] The method of fluorescent staining for extracellular vesicles according to [9] above,
wherein the sample includes cells, and
wherein the method measures the extracellular vesicles in the cells to evaluate the state of the cells.
   [13] The method of fluorescent staining for extracellular vesicles according to [9] above,
wherein the sample includes foods or a beverages, and
wherein the method measures the extracellular vesicles in the foods or the beverages to evaluate the foods or the beverages.
   [14] The method of fluorescent staining for extracellular vesicles according to [9] above,
wherein the sample includes tissues collected from organisms, and
wherein the method measures the extracellular vesicles in the collected tissues to evaluate the tissues.

### Advantageous Effects of Invention

Staining extracellular vesicles with the compound disclosed in the present application enables size measurement such as liquid chromatography.

### BRIEF DESCRIPTION OF DRAWINGS

FIG. 1a is a graph illustrating a result of analysis on stained milk-derived extracellular vesicles by a high-performance liquid chromatography gel filtration system in Comparative example 1.

FIG. 1b represents graphs illustrating results of analysis on stained milk-derived extracellular vesicles by the high-performance liquid chromatography gel filtration system in Example 10.

FIG. 1c represents graphs illustrating results of analysis on stained milk-derived extracellular vesicles by the high-performance liquid chromatography gel filtration system in Example 10.

FIG. 1d represents graphs illustrating results of analysis on stained milk-derived extracellular vesicles by the high-performance liquid chromatography gel filtration system in Example 10.

FIG. 1e represents graphs illustrating results of analysis on stained milk-derived extracellular vesicles by the high-performance liquid chromatography gel filtration system in Example 10.

FIG. 1f is a graph illustrating a result of analysis on stained milk-derived extracellular vesicles by the high-performance liquid chromatography gel filtration system in Example 10.

FIG. 2a is a graph illustrating a result of stained milk-derived extracellular vesicles being heat-treated and analyzed by the high-performance liquid chromatography gel filtration system in Comparative example 2.

FIG. 2b represents graphs illustrating results of stained milk-derived extracellular vesicles being heat-treated and analyzed by the high-performance liquid chromatography gel filtration system in Example 11.

FIG. 2c represents graphs illustrating results of stained milk-derived extracellular vesicles being heat-treated and analyzed by the high-performance liquid chromatography gel filtration system in Example 11.

FIG. 3 represents graphs illustrating results of stained various lactic acid bacteria-derived extracellular vesicles being heat-treated and analyzed by the high-performance liquid chromatography gel filtration system in Example 12.

FIG. 4 is a graph illustrating a result of successful size separation of stained milk-derived and stained lactic acid bacteria-derived extracellular vesicles by the high-performance liquid chromatography gel filtration system in Example 13.

FIG. 5a is a graph illustrating a result of analysis on stained lactic acid bacteria (Yakult)-derived extracellular vesicles by the high-performance liquid chromatography gel filtration system in Comparative example 3.

FIG. 5b represents graphs illustrating results of analysis on stained lactic acid bacteria (Yakult)-derived extracellular vesicles by the high-performance liquid chromatography gel filtration system in Example 14.

FIG. 5c represents graphs illustrating results of analysis on stained lactic acid bacteria (Yakult)-derived extracellular vesicles by the high-performance liquid chromatography gel filtration system in Example 14.

FIG. 5d represents graphs illustrating results of analysis on stained lactic acid bacteria (Yakult)-derived extracellular vesicles by the high-performance liquid chromatography gel filtration system in Example 14.

FIG. 5e represents graphs illustrating results of analysis on stained lactic acid bacteria (Yakult)-derived extracellular vesicles by the high-performance liquid chromatography gel filtration system in Example 14.

FIG. 5f is a graph illustrating a result of analysis on stained lactic acid bacteria (Yakult)-derived extracellular vesicles by the high-performance liquid chromatography gel filtration system in Example 14.

FIG. 6 is a photograph confirming that milk-derived extracellular vesicles labeled with GIF-2276 can be tracked in introduced cells in Example 15.

FIG. 7 is a photograph confirming that lactic acid bacteria-derived extracellular vesicles labeled with GIF-2276 can be tracked in introduced cells in Example 16.

FIG. 8 is a graph illustrating a result of analysis by the high-performance liquid chromatography gel filtration system on milk-derived extracellular vesicles stained with GIF-2250 disclosed in Patent Literature 1 and GIF-2276 synthesized in Example 1 in Example 17 and Comparative example 4.

FIG. 9 represents graphs illustrating that, in Example 17 and Comparative example 4, there is a correlation between detection of extracellular vesicles by a plate reader using GIF-2250 disclosed in Patent Literature 1 and detection of extracellular vesicles by gel filtration using GIF-2276 synthesized in Example 1.

FIG. 10 is a graph illustrating a result of analysis, in Example 18, on extracellular vesicles contained in milk having different degrees of freshness using GIF-2276 synthesized in Example 1.

### DESCRIPTION OF EMBODIMENTS

### Embodiment of Compound

Compounds according to the embodiments will be described below.

The compounds according to the embodiments are characterized by being a compound expressed by the following Formula (1). Each compound expressed by Formula (1) specifically conjugates with extracellular vesicles and thus emits fluorescence. Note that the term “extracellular vesicle” in the present specification represents a collective term of vesicles secreted from cells such as exosomes, microvesicles, apoptotic bodies, or the like.

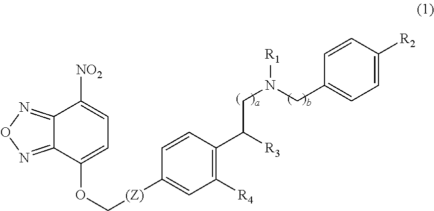

In Formula (1), R₁ represents H, a C₁-C₆ alkyl group, a hydroxy group, an amino group, or a carboxy group. The C₁-C₆ alkyl group may be linear, branched, or cyclic. For example, the C₁-C₆ alkyl group may be a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a neopentyl group, an n-hexyl group, an isohexyl group, a sec-hexyl group, a cyclopropyl group, a cyclobutyl group, a cyclopentyl group, a cyclohexyl group, or the like.

R₂ represents H, a C₁-C₁₈ alkyl group, a C₁-C₁₈ alkoxy group, NO₂, or N(CH₃)₂. The C₁-C₁₈ alkyl group may be linear, branched, or cyclic. The C₁-C₁₈ alkyl group may be, for example, a methyl group, an ethyl group, an n-propyl group, an isopropyl group, an n-butyl group, an isobutyl group, a sec-butyl group, a tert-butyl group, an n-pentyl group, an isopentyl group, a sec-pentyl group, a neopentyl group, an n-hexyl group, an isohexyl group, a sec-hexyl group, an n-heptyl group, an isoheptyl group, a sec-heptyl group, an n-octyl group, an isooctyl group, a sec-octyl group, a nonyl group, a decyl group, an undecyl group, a dodecyl group, a tridecyl group, a tetradecyl group, a pentadecyl group, a hexadecyl group, a heptadecyl group, an octadecyl group, a cyclopropyl group, a cyclobutyl group, a cyclopentyl group, a cyclohexyl group, a cycloheptyl group, a cyclooctyl group, a cyclononyl group, a cyclodecyl group, a cycloundecyl group, a cyclododecyl group, a cyclotridecyl group, a cyclotetradecyl group, a cyclopentadecyl group, a cyclohexadecyl group, a cycloheptadecyl group, a cyclooctadecyl group, or the like. The C₁-C₁₈ alkoxy group may be saturated or unsaturated and may have an aromatic ring. Further, the C₁-C₁₈ alkoxy group may be linear, branched, or cyclic. The C₁-C₁₈ alkoxy group may be, for example, a methoxy group, an ethoxy group, a propoxy group, an isopropoxy group, a cyclopropoxy group, an n-butoxy group, an isobutoxy group, a tert-butoxy group, a pentyloxy group, a hexyloxy group, an octyloxy group, a nonaoxy group, a decaneoxy group, an undecaneoxy group, a dodecaneoxy group, a tridecaneoxy group, a tetradecaneoxy group, a pentadecaneoxy group, a hexadecaneoxy group, a heptadecaneoxy group, an octadecaneoxy group, a vinyloxy group, an allyloxy group, a 1-propenyloxy group, an isopropenyloxy group, a 1-butenyloxy group, a 2-butenyloxy group, a 3-butenyloxy group, a 1,3-butanedienyloxy group, a 1-pentenyloxy group, a 2-pentenyloxy group, a 3-pentenyloxy group, a 4-pentenyloxy group, a hexynyloxy group, a hexidinyloxy group, a heptinyloxy group, a heptidinyloxy group, an octinyloxy group, an octidinyloxy group, a noninyloxy group, a nonidinyloxy group, a decynyloxy group, a decidinyloxy group, an undecynyloxy group, an undecidinyloxy group, a dodecyloxy group, a dodecidinyloxy group a tridecynyloxy group, a tridecidinyloxy group, a tetradecynyloxy group, a tetradecidinyloxy group, a pentadecynyloxy group, a pentadecidinyloxy group, a hexadecynyloxy group, a hexadecidinyloxy group, a heptadecynyloxy group, a heptadecidinyloxy group an octadecynyloxy group, an octadecidinyloxy group, a phenoxy group, a naphthoxy group, an anthryloxy group, a methylphenoxy group, a dimethylphenoxy group, a trimethylphenoxy group, an ethylphenoxy group, a diethylphenoxy group, a triethylphenoxy group, a propylphenoxy group, a butylphenoxy group, a methylnaphthoxy group, a dimethylnaphthoxy group, a trimethylnaphthoxy group, a methylanthryloxy group, an ethylanthryloxy group, a benzyloxy group, a phenethyloxy group, a naphthyl methyloxy group, a fluorenyl methyloxy group, or the like.

R₃ and R₄ each independently represent H or CH₃, and R₃ and R₄ may be the same as or different from each other. Note that, R₃ and R₄ may be bonded to each other to form a ring, and in such a case, R₃ and R₄ are CH₂.

Herein, “a” represents 0 or 1, and “b” represents 1, 2, or 3.

Z represents a linker that links the fluorescent group located on the left side from (Z) and the chemical structure on the right side from (Z) in Formula (1) to each other. The linker (Z) is formed of bonded 0 to 12 chain molecules formed of elements selected from a group consisting of C, O, and N, and the chain molecules may include a cyclic structure or a branched chain. The linker (Z) will be more specifically described with reference to an example in which three chain molecules (x, y, z) are bonded into (-x-y-z-), for example.

Each of the chain molecules x, y, and z may be any element selected from C, O, and N. In such a case, the selected elements may be the same or different. For example, the linker may be formed of the same elements such as -c-c-c- or may be formed of different elements being bonded such as -c-n-c-.

One or more of any of the chain molecules x, y, and z may be formed of two or more elements as long as the one or more chain molecules are formed of elements selected from the group consisting of C, O, and N. For example, any one or more chain elements may include a cyclic structure formed of elements selected from the group consisting of C, O, and N. The cyclic structure may be a monocyclic ring, bicyclic rings, or tricyclic rings. Further, the bonding between ring atoms may be fully saturated or may be unsaturated.

Further, any one or more chain molecules may include a branched chain formed of elements selected from the group consisting of C, O, and N. The branched chain is not particularly limited as long as it can be substituted with C, O, N elements forming -x-y-z-. The chain molecules may be a linear carbon chain such as methyl, ethyl, or the like or may be C bonded to “═O” (ketone as a chain molecule).

More specific examples of the compound expressed by Formula (1) may be compounds expressed by Formula (2) and compounds expressed by Formula (3) below.

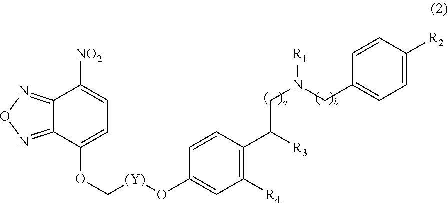

In Formula (2), R₁ represents H or CH₃. R₂ represents H, CH₃, OCH₃, or N(CH₃)₂. R₃ and R₄ each independently represent H or CH₃. Note that R₃ and R₄ may be bonded to each other to form a ring, and in such a case, R₃ and R₄ are CH z. Herein, “a” represents 0 or 1, and “b” represents 1, 2, or 3. Y represents a C₁-C₅ alkyl group, an ether chain expressed by Formula (2a) below, an ether chain including amido expressed by Formula (2b) below, or triazole expressed by Formula (2c) below. Note that the symbols “-” at both terminals in Formulas (2a), (2b), and (2c) indicated below correspond to the symbols “-” at both terminals of “—(Y)—” in Formula (2).

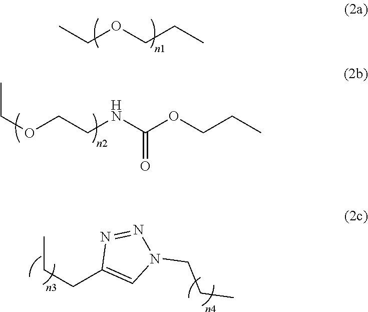

In Formula (2a), n1 represents an integer of 1 to 3. In Formula (2b), n2 represents an integer of 1 to 3. In Formula (2c), n3 represents an integer of 0 to 6, and n4 represents an integer of 0 to 3. Further, Formulas (2a), (2b), and (2c) may be replaced with “—(Y)—” in the direction depicted in the sheet or may be left-right-inverted and replaced with “—(Y)—”. In the present application, the description of Formulas (2a), (2b), and (2c) includes both the replacement with “—(Y)—” in the direction depicted in the sheet and the left-right inversion and replacement with “—(Y)—”.

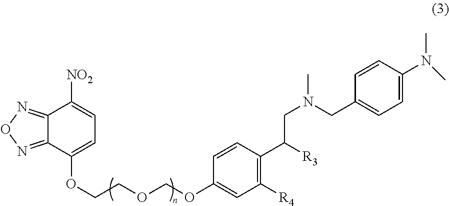

In Formula (3), R₃ and R₄ each independently represent H or CH₃. Note that R₃ and R₄ may be bonded to each other to form a ring and, in such a case, R₃ and R₄ are CH₂. Herein, n represents an integer of 0 to 3.

While specific examples of the compounds expressed by Formula (2) and the compounds expressed by Formula (3) are indicated below, the compounds are not limited to these illustrated compounds.

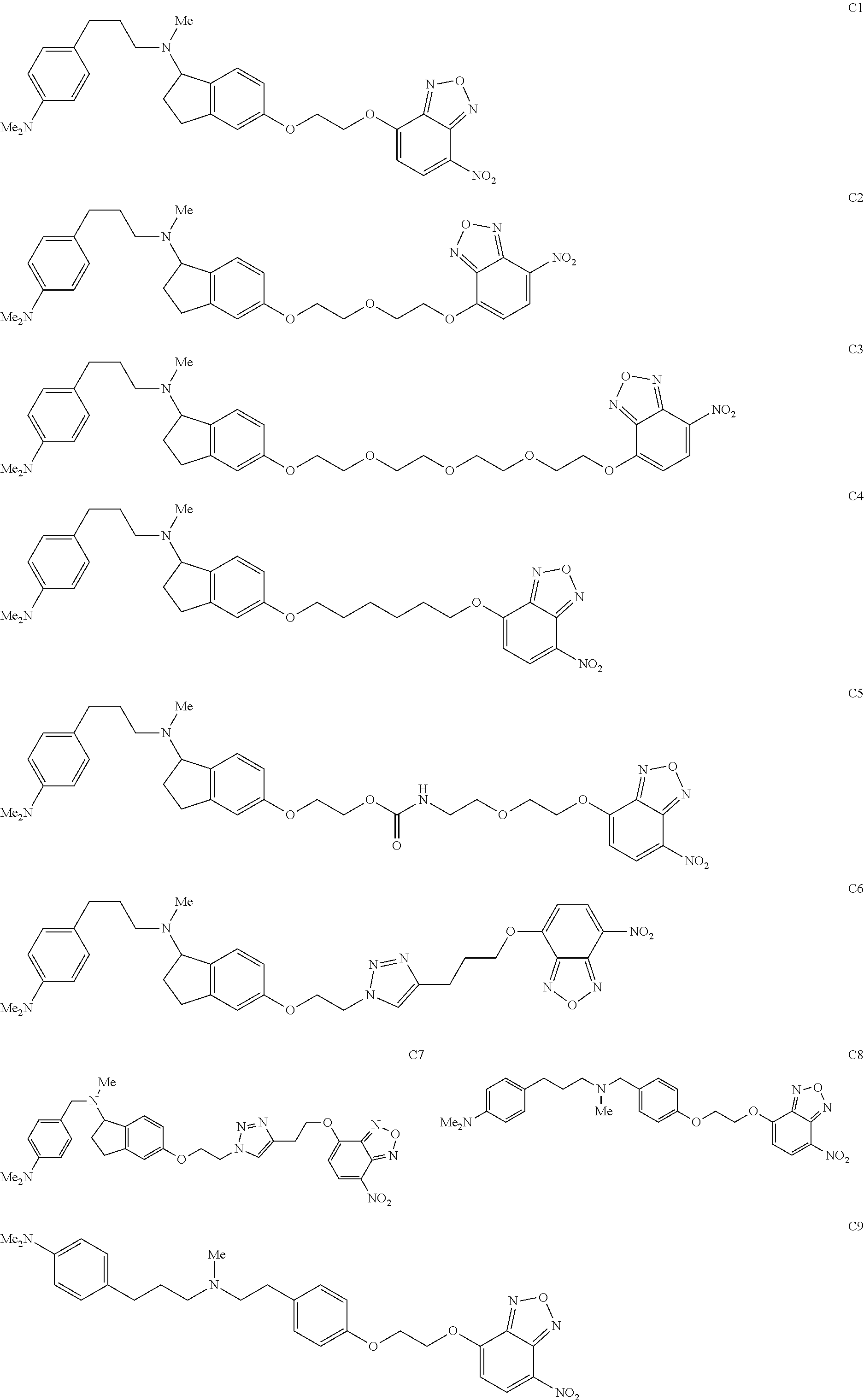

The compound expressed by Formula (1) does not emit fluorescence by its self. In contrast, in Examples described below, because of the fact that fluorescence was observed when the compound expressed by Formula (1) was mixed with extracellular vesicles and that fluorescence was also observed in size separation by liquid chromatography, it is considered that:
the compound expressed by Formula (1) changes its structure due to a reaction with extracellular vesicles; and
the compound is firmly bonded to extracellular vesicles rather than being ionically bonded to extracellular vesicles by electrostatic attraction.

From a view point of the structure of Formula (1), it is considered that, upon contact with extracellular vesicles,

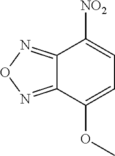

which is a fluorescent group of a compound expressed by Formula (1) is detached from the compound disclosed in the present application, and the moiety of —O— of the fluorescent group is covalently bonded to the amino group of lysine of extracellular vesicles.

### Embodiment of Extracellular Vesicle Staining Agent

The compound according to the embodiment described above can also be dissolved in a solvent and thus be used as an extracellular vesicle staining agent. The solvent is not particularly limited as long as the solvent can dissolve the compound expressed by Formula (1) therein. The solvent may be, for example, acetone, DMSO, ethanol, an aqueous solution thereof, or the like. It can also be used in an aqueous solution or buffer solution itself.

The extracellular vesicle staining agent according to the embodiment may optionally and additionally contain other fluorescence compounds that fluorescently stain elements different from extracellular vesicles such as mitochondria, cell membranes, or the like, for example, other than the compound expressed by Formula (1). By using the compound expressed by Formula (1) and a fluorescent compound, it is possible to stain a plurality of elements at the same time. Further, with the interaction between the compound expressed by Formula (1) and the fluorescent compound, it is possible to sensitize the compound expressed by Formula (1) and improve specificity of the compound expressed by Formula (1) to cellular vesicles.

### Embodiment of Extracellular Vesicle Staining Agent

A method of fluorescent staining for extracellular vesicles according to the embodiment includes at least a staining step of staining extracellular vesicles by using the compound expressed by Formula (1) or an extracellular vesicle staining agent and a detection step of detecting the stained extracellular vesicles in a sample and optionally and additionally includes an evaluation step of evaluating the sample after the detection step.

The staining step is to fluorescently stain extracellular vesicles with the compound expressed by Formula (1). For example, when staining is performed on purified extracellular vesicles, the compound expressed by Formula (1) can be added to an extracellular vesicle solution. Note that purification of extracellular vesicles can be performed by a known method. Further, the culture solution for the cultured cells may be collected, and the compound expressed by Formula (1) may be added to the collected culture solution. When extracellular vesicles are incorporated into cells and tracking thereof is then performed, the compound expressed by Formula (1) can be added to a solution containing extracellular vesicles.

In the staining step, for the amount of the compound expressed by Formula (1) to be added, the final concentration after the addition may be in a range of 0.01 μM to 10 μM, preferably in a range of 0.3 μM to 3 μM.

The detection step is to irradiate the sample with excitation light corresponding to a fluorescent group of the compound expressed by Formula (1) and detect fluorescence emitted from the compound expressed by Formula (1) conjugated with extracellular vesicles in the sample. For irradiation of excitation light, the same irradiation means as used in general fluorescence detection may be used, and a predetermined wavelength may be, for example, selected from a laser beam source of a fluorescence microscope as required. Further, in a fluorescent detector connected to high-performance liquid chromatography, fluorescent excitation and fluorescent wavelengths can be set. While depending on the fluorescent group of the compound expressed by Formula (1), the fluorescence may be observed through image capturing by a camera or the like, even when it is not possible to sufficiently observe fluorescence by visual observation through a lens barrel of the fluorescence microscope. A filter that selectively passes light having a predetermined wavelength may be used as required.

Further, the method of fluorescent staining for extracellular vesicles optionally and additionally includes an evaluation step of evaluating a sample. The evaluation step is to evaluate the sample by using extracellular vesicles detected by the compound expressed by Formula (1). While what to evaluate depends on a sample, it is possible to evaluate cosmetics, pharmaceutical products, foods, or the like, for example. Moreover, it is possible to detect intracellular vesicles to evaluate the degree of cell differentiation or detect exosomes in body fluids to evaluate diseases or the like.

For example, when cosmetics or pharmaceutical products are evaluated, extracellular vesicles are labeled with the compound expressed by Formula (1) in advance, incorporation of the labeled extracellular vesicles into the cells is detected and observed in accordance with the presence of the cosmetics or the pharmaceutical products, and thereby the cosmetics or the pharmaceutical products can be evaluated. For example, it is possible to fluorescently label melanosomes, which are vesicles to store melanin, and evaluate pigmentation or skin diseases involved therewith based on incorporation of the labeled substances into keratinocytes. Further, it is possible to evaluate a food by measuring extracellular vesicles included in the food. For example, it is possible to predict a food function from efficiency of miRNA incorporation into vesicles that affect intestinal functions.

Size separation can be performed by gel filtration on the extracellular vesicles labeled with the compound expressed by Formula (1). When the separation is performed by a high-performance liquid chromatography gel filtration system, a column such as Shodex OHpak SB-807 (by Showa Denko), TSKgel G7000HHR (by TOSOH), Gen 2 qEV original/70 nm or Gen 2 qEV original/35 nm (by iZQN), or a combination of these columns can be used.

Further, after the separation by the gel filtration system, extracellular vesicles can also be fractionated.

The compound and the method of fluorescent staining for extracellular vesicles according to the embodiments achieve the following advantageous effects.

(1) The compound expressed by Formula (1) does not emit fluorescence by its self. Thus, removal of unreacted substances is unnecessary.
(2) The compound expressed by Formula (1) is firmly bonded to extracellular vesicles. Thus, while the extracellular vesicles stained with the compound disclosed in Patent Literature 1 are unable to be used for size separation by liquid chromatography, the compound expressed by Formula (1) disclosed in the present application can be used even for size separation where a fluid pressure is applied, such as liquid chromatography. For example, different types of dairy products have different extracellular vesicle sizes. When size separation of extracellular vesicles is possible, the size separation can be used for use such as identification of food types.
(3) By using the compound expressed by Formula (1) to detect extracellular vesicles, it is possible to evaluate cosmetics, pharmaceutical products, foods, degree of cell differentiation, diseases, or the like.

Although embodiments disclosed in the present application will be specifically described with Examples presented below, these Examples are merely provided for describing the embodiment. These Examples are merely provided for illustration of the embodiment, which are intended neither to limit the scope of the invention disclosed in the present application nor to express limitation of the same.

### EXAMPLE

### [Synthesis of Compound]

### Example 1

Compound 1 (C1) was synthesized in the procedure described below.

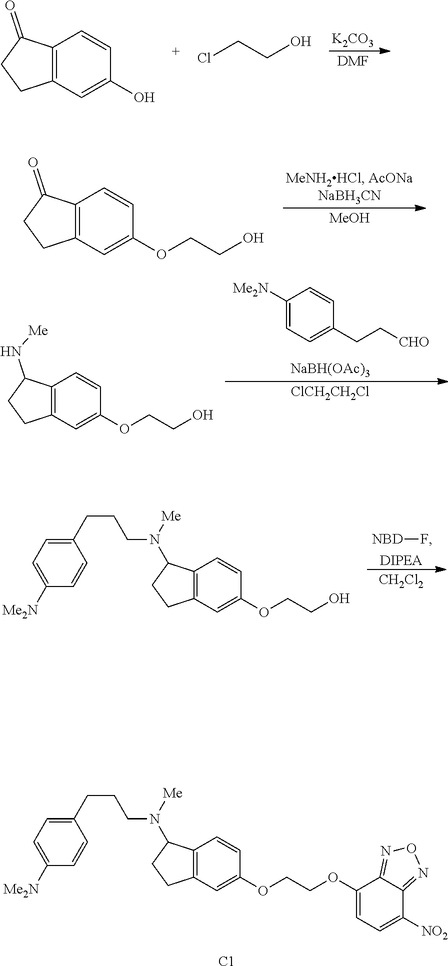

### 5-(2-hydroxyethoxy)-1-indanone

Potassium carbonate (829 mg) and 2-chloroethanol (1 mL) were added to N,N-dimethylformamide (15 mL) solution containing 5-hydroxy-1-indanone (741 mg, 5 mmol), and the mixture was heated and stirred at 100° C. for 30 minutes. The reaction liquid was allowed to be cooled to room temperature and then poured in water, and the resulting product was extracted with dichloromethane and dried with anhydrous sodium sulfate. A mixed solution of ethyl acetate/hexane at 1:4 was added to the residue obtained by concentrating the extracted liquid under reduced pressure, the deposited precipitate was filtered out, and a light yellow solid of the compound as titled above (699 mg, yield of 73%) was yielded.

¹H NMR (400 MHz, CDCl₃) δ: 2.68 (m, 2H, COCH₂), 3.09 (t, J=6 Hz, 2H, ArCH₂), 3.98-4.05 (br, 2H, CH₂OH), 4.17 (t, J=4.6 Hz, 2H, ArOCH₂), 6.9-6.95 (complex, 2H, ArH), 7.70 (d, J=8.7 Hz, 2H, ArH)

### 2-{[1-({3-[4-(dimethylamino)phenyl]propyl}(methyl)amino)indan-5-yl]oxy}-1-Ethanol

Methylamine hydrochloride (203 mg) and sodium acetate (246 mg) were added at room temperature to methanol (5 mL) solution containing 5-(2-hydroxyethoxy)-1-indanone (192 mg, 1 mmol), and the mixture was stirred for 30 minutes. Subsequently, sodium cyanoborohydride (126 mg) was added to the reaction solution, and the mixture was heated to reflux for 24 hours. The reaction liquid was allowed to be cooled to room temperature and then poured in water, extracted with dichloromethane, and dried with anhydrous sodium sulfate. The extracted liquid was concentrated under reduced pressure to obtain a light yellow solid of 2-{[1-(methylamino)indan-5-yl]oxy}-1-ethanol (210 mg). The obtained methylamine compound (210 mg) and 3-[4-(dimethylamino)phenyl]propanal (177 mg) were dissolved in dichloroethane (5 mL), sodium triacetoxyborohydride (371 mg) was added thereto, and the mixture was stirred at room temperature for 23 hours. The reaction solution was poured in a saturated sodium hydrogen carbonate aqueous solution, extracted with dichloromethane, and dried with anhydrous sodium sulfate. The residue obtained by concentrating the extracted liquid under reduced pressure was purified with column chromatography (silica gel, acetone), and a light yellow oily compound as titled above (249 mg, 68%) was yielded.

¹H NMR (400 MHz, MeOH-d4) δ: 1.7-1.88 (m, 2H, CH₂CH₂CH₂), 2.02-2.1 (m, 2H, ArCH₂CH₂CH), 2.16 (s, 3H, NCH₃), 2.35-2.45 (m, 2H, ArCH₂), 2.45-2.54 (br t, NCH₂), 2.71-2.92 (m, 2H, ArCH₂), 2.86 (s, 6H, N(CH₃)₂), 3.85 (t, J=4.8 Hz, 2H, CH₂OH), 4.01 (t, J=4.8 Hz, 2H, ArOCH₂), 4.33 (br t, J=6.4 Hz, 1H, ArCHNH), 6.72 (d, J=8.2 Hz, 2H, ArH), 6.77 (d, J=8.7 Hz, 1H, ArH), 6.79 (s, 1H, ArH), 7.0 (d, J=8.7 Hz, 2H, ArH), 7.21 (d, J=8.2 Hz, 1H, ArH)

Compound 1 (C1, Hereafter, which May be Referred to as “GIF-2276”):

2-{[1-({3-[4-(dimethylamino)phenyl]propyl}(methyl)amino)indan-5-yl]oxy}-1-ethanol (36 mg, 97 mol) was dissolved in dichloromethane (1 mL), 4-fluoro-7-nitrobenzofurazan (NBD-F, 18 mg) and N,N-diisopropylethylamine (34 μL) were added thereto, and the mixture was stirred at room temperature for 43 hours. The residue obtained by concentrating the reaction liquid under reduced pressure was purified with PLC (silica gel, acetone/methanol=85/15), and a brown solid of Compound 1 (20 mg, 39%) was yielded.

¹H NMR (400 MHz, CDCl₃) δ: 1.78-1.88 (m, 2H, CH₂CH₂CH₂), 2.04-2.13 (br m, 2H, ArCH₂CH₂CH), 2.22 (s, 3H, NCH₃), 2.46-2.61 (complex, 4H, ArCH₂ and NCH₂), 2.76-2.83 (m, 1H, ArCH₂), 2.86-2.94 (m, 1H, ArCH₂), 2.9 (s, 6H, N(CH₃)₂), 4.43-4.5 (br, 1H, ArCHN), 4.50 (t, J=3 Hz, 2H, OCH₂CH₂Ar), 4.78 (br t, J=3 Hz, 2H, ArOCH₂), 6.69 (d, J=5.8 Hz, 2H, ArH), 6.76-6.8 (complex, 2H ArH), 6.83 (d, J=5.5 Hz, 1H, ArH), 7.05 (d, J=5.8 Hz, 2H, ArH), 7.26-7.31 (1H, ArH), 8.57 (d, J=5.5 Hz, 1H, ArH)

### Example 2

Compound 2 (C2) was synthesized in the procedure described below.

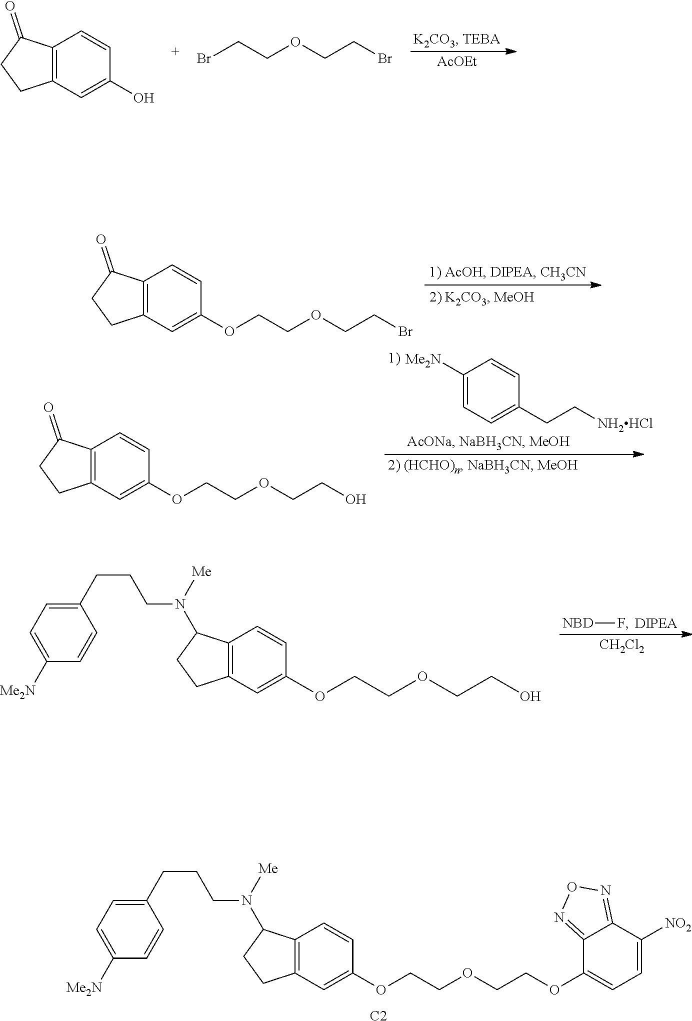

### 5-[2-(2-bromoethoxy)ethoxy]-1-indanone

Potassium carbonate (1.47 g), bis(2-bromoethyl) ether (1.82 mL), and benzyltriethylammonium chloride (TEBA) (76 mg) were sequentially added to ethyl acetate (20 mL) solution containing 5-hydroxy-1-indanone (435 mg, 2.94 mmol), and the mixture was heated to reflux for 24 hours. The reaction liquid allowed to be cooled to room temperature was poured in ice water, extracted with ethyl acetate, washed with a saturated saline solution, and dried with anhydrous sodium sulfate. The residue obtained by concentrating the extracted liquid under reduced pressure was purified with column chromatography (silica gel, hexane/ethyl acetate=1/1), and a white solid of the compound as titled above (654 mg, 74%) was yielded.

¹H NMR (400 MHz, CDCl₃) δ: 2.66 (m, 2H, COCH₂), 3.07 (br t, J=6 Hz, 2H, ArCH₂), 3.49 (t, J=6.2 Hz 2H, BrCH₂), 3.88 (t, J=6.4 Hz, 2H, OCH₂), 3.90 (t, J=5 Hz, 2H, OCH₂), 4.21 (t, J=5 Hz, 2H, ArOCH₂), 6.89-6.93 (br m, 2H, ArH), 7.68 (d, J=9.2 Hz, 1H, ArH)

### 5-[2-(2-hydroxyethoxy)ethoxy]-1-indanone

Acetic acid (0.43 mL) and N,N-diisopropylethylamine (1.45 mL) were added to acetonitrile (12 mL) solution containing 5-[2-(2-bromoethoxy)ethoxy]-1-indanone (378 mg, 1.26 mmol), and the mixture was heated to reflux for 26 hours. The reaction liquid was allowed to be cooled to room temperature and then poured in a saturated sodium hydrogen carbonate aqueous solution, extracted with ethyl acetate, washed with dilute hydrochloric acid and saline solution, and dried with anhydrous sodium sulfate. A light brown oily 5-[2-(2-acetoxyethoxy)ethoxy]-1-indanone (385 mg) obtained by concentrating the extracted liquid under reduced pressure was dissolved in methanol (5 mL), potassium carbonate (83 mg) was added thereto, and the mixture was stirred at room temperature for 4 hours. Water was poured in the reaction liquid, and the resulting product was extracted with dichloromethane and dried with anhydrous sodium sulfate. The extracted liquid was concentrated under reduced pressure, and a white solid of the compound as titled above (298 mg, quantitative) was yielded.

¹H NMR (400 MHz, CDCl₃) δ: 2.66-2.69 (m, 2H, COCH₂), 3.09 (t, J=4 Hz, 2H, ArCH₂), 3.67-3.7 (m 2H, OCH₂), 3.77-3.8 (m, 2H, OCH₂), 3.89-3.92 (m, 2H, OCH₂), 4.22 (t, J=3 Hz, 2H, ArOCH₂), 6.92 (s, 1H, ArH), 6.92-6.94 (m, 2H, ArH), 7.69 (d, J=6.2 Hz, 1H, ArH)

### 2-(2-{[1-({3-[4-(dimethylamino)phenyl]propyl}(methyl)amino)indan-5-yl]oxy]ethoxy)-1-ethanol

5-[2-(2-hydroxyethoxy)ethoxy]-1-indanone (59 mg, 0.25 mmol) and 4-(3-aminopropyl)-N,N-dimethylaniline hydrochloride (64 mg) were dissolved in methanol (1 mL), sodium acetate (25 mg) was added thereto, and the mixture was stirred at room temperature for 30 minutes. Subsequently, sodium cyanoborohydride (24 mg) and acetic acid (17 μL) were added to the mixture to be allowed to react at room temperature for 42 hours. A saturated sodium hydrogen carbonate aqueous solution was added to the reaction liquid, extracted with ethyl acetate, and dried with anhydrous sodium sulfate. A light yellow solid (119 mg) obtained by concentrating the extracted liquid under reduced pressure was dissolved in methanol (1 mL), paraformaldehyde (38 mg) and sodium cyanoborohydride (32 mg) were added thereto, and the mixture was stirred at room temperature for 22 hours. A saturated sodium hydrogen carbonate aqueous solution was added to the reaction liquid, extracted with ethyl acetate, and dried with anhydrous sodium sulfate. The residue obtained by concentrating the extracted liquid under reduced pressure was purified with column chromatography (silica gel, acetone/methanol=9/1), and a colorless oily compound as titled above (57 mg, yield of 55%) was yielded.

¹H NMR (400 MHz, CDCl₃) δ: 1.73-1.85 (m, 2H, CH₂CH₂CH₂), 1.99-2.08 (m, 2H, ArCH₂CH₂CH), 2.16 (s, 3H, NCH₃), 2.4-2.64 (complex, 4H, ArCH₂ and NCH₂), 2.71-2.81 (m, 1H, ArCH₂), 2.83-2.9 (m, 1H, ArCH₂), 2.90 (s, 6H, N(CH₃)₂), 3.68 (t, J=4.3 Hz, 2H, OCH₂), 3.76 (t, J=4.3 Hz, 2H, OCH₂), 3.87 (t, J=4.8 Hz, 2H, OCH₂), 4.13 (t, J=4.8 Hz, 2H, ArOCH₂), 4.37 (br t, J=7.1 Hz, 1H, ArCHN), 6.69 (d, J=8.7 Hz, 2H, ArH), 6.76 (s, 1H, ArH), 6.75-6.79 (1H, ArH), 7.06 (d, J=8.7 Hz, 2H, ArH), 7.23-7.26 (1H, ArH)

Compound 2 (C2, Hereafter, which May be Referred to as “GIF-2277”):

NBD-F (10 mg) and N,N-diisopropylethylamine (12 μL) were added to dichloromethane (0.5 mL) solution containing 2-(2-{[1-({3-[4-(dimethylamino)phenyl]propyl}(methyl)amino)indan-5-yl]oxy]ethoxy)-1-ethanol (23 mg, 55 mol), and the mixture was stirred at room temperature for 48 hours. A saturated sodium hydrogen carbonate aqueous solution was added to the reaction liquid, extracted with dichloromethane, and dried with anhydrous sodium sulfate. The residue obtained by concentrating the extracted liquid under reduced pressure was purified with PLC (silica gel, acetone/methanol=9/1), and Compound 2 (16.2 mg, 51%) was yielded.

¹H NMR (400 MHz, CDCl₃) δ: 1.75-1.86 (br, 2H, CH₂CH₂CH₂), 2.0-2.1 (m, 2H, ArCH₂CH₂CH) 2.17 (s, 3H, NCH₃), 2.43-2.64 (complex, 4H, ArCH₂ and NCH₂), 2.7-2.8 (m, 1H, ArCH₂) 2.81-2.9 (m, 1H, ArCH₂), 2.90 (s, 6H, N(CH₃)₂), 3.94 (t, J=4.4 Hz, 2H, OCH₂) 4.09-4.15 (complex, 4H, OCH₂), 4.37-4.44 (br, 1H, ArCHN), 4.6 (t, J=4.6 Hz, 2H, ArOCH₂) 6.66-6.73 (complex, 4H, ArH), 6.76 (d, J=8.2 Hz, 1H, ArH), 7.05 (d, J=8.2 Hz, 2H, ArH) 7.21-7.26 (1H, ArH), 8.47 (d, J=8.2 Hz, 1H, ArH)

### Example 3

Compound 3 (C3) was synthesized in the procedure described below.

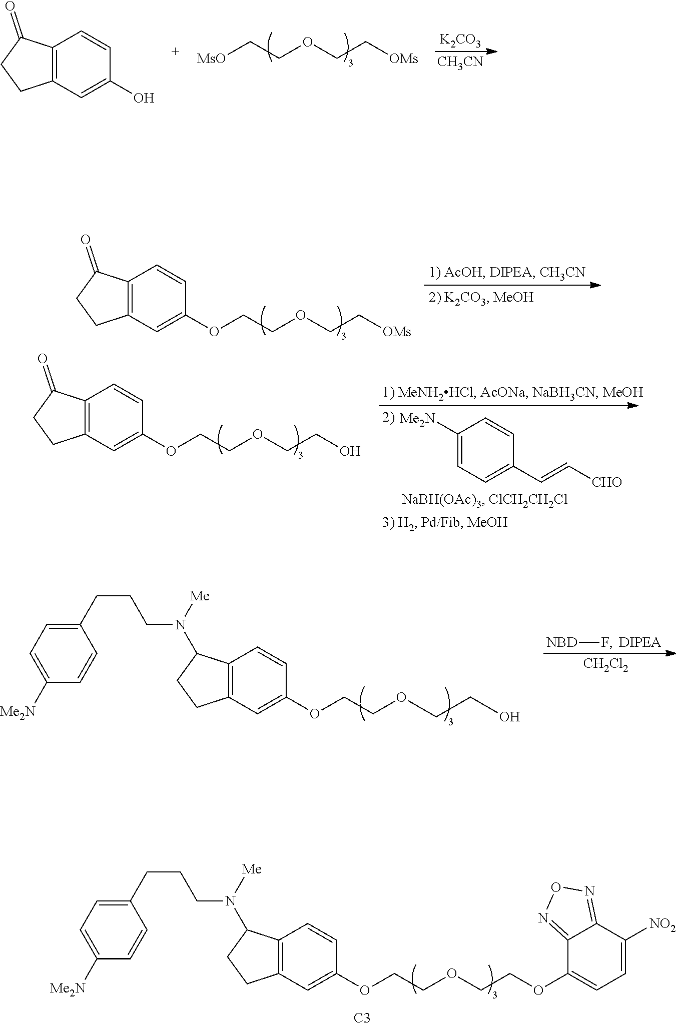

### 5-[(11-acetoxy-3,6,9-trioxaundecyl)oxy]-1-indanone

Potassium carbonate (1.38 g) and tetraethylene glycol dimesylate (2.63 g) were added to acetonitrile (10 mL) solution containing 5-hydroxy-1-indanone (741 mg, 5 mmol), and the mixture was heated to reflux for 22 hours. The reaction liquid was allowed to be cooled to room temperature and then poured in a saline solution, and the resulting product was extracted with ethyl acetate and dried with anhydrous sodium sulfate. The extracted liquid was concentrated under reduced pressure to obtain light brown oily 5-[(11-mesyloxy-3,6,9-trioxaundecyl)oxy]-1-indanone. The obtained monomesylate was dissolved in acetonitrile (20 mL), acetic acid (2.86 mL) and N,N-diisopropylethylamine (8.7 mL) were added thereto, and the mixture was heated to reflux for 30 hours. The reaction liquid was allowed to be cooled to room temperature and then poured in a saturated sodium hydrogen carbonate aqueous solution, the resulting product was extracted with ethyl acetate, washed with dilute hydrochloric acid and a saline solution, and dried with anhydrous sodium sulfate. The residue obtained by concentrating the extracted liquid under reduced pressure was purified with column chromatography (silica gel, ethyl acetate), and a colorless oily compound as titled above (1.02 g, 56%) was yielded.

¹H NMR (400 MHz, CDCl₃) δ: 2.65-2.7 (m, 2H, COCH₂), 3.08 (br t, J=6 Hz, 2H, ArCH₂), 3.65-3.76 (complex, 10H, OCH₂), 3.89 (t, J=4.8 Hz, 2H, OCH₂) 4.19-4.24 (complex 4H, ArOCH₂ and CH₃COOCH₂), 6.91 (s, 1H, ArH), 6.91-6.94 (1H, ArH), 7.68 (d, J=9.2 Hz, 1H, ArH)

### 5-[(11-hydroxy-3,6,9-trioxaundecyl)oxy]-1-indanone

5-[(11-acetoxy-3,6,9-trioxaundecyl)oxy]-1-indanone (1.02 g, 2.78 mmol) was dissolved in methanol (10 mL), potassium carbonate (38 mg) was added thereto, and the mixture was stirred at room temperature for 1 hour. The reaction liquid was concentrated under reduced pressure and then poured in water, extracted with dichloromethane, and dried with anhydrous sodium sulfate. The extracted liquid was concentrated under reduced pressure to obtain a light yellow oily compound as titled above (781 mg). This compound was not purified and used in the next step.

¹H NMR (400 MHz, CDCl₃) δ: 2.65-2.7 (m, 2H, COCH₂), 3.09 (t, J=6 Hz, 2H, ArCH₂), 3.61 (t, J=4.6 Hz, 2H, OCH₂), 3.64-3.77 (complex, 10H, OCH₂), 3.89 (t, J=4.7 Hz, 2H, OCH₂), 4.21 (t, J=4.8 Hz, 2H, ArOCH₂), 6.92 (s, 1H, ArH), 6.91-6.95 (1H, 1H, ArH), 7.68 (d, J=9.6 Hz, 1H, ArH)

### 11-{[1-({3-[4-(dimethylamino)phenyl]propyl}(methyl)amino)indan-5-yl]oxy}-1-(3,6,9-trioxa)undecanol

Methylamine hydrochloride (203 mg) and sodium acetate (246 mg) were added at room temperature to methanol (5 mL) solution containing 5-[(11-hydroxy-3,6,9-trioxaundecyl)oxy]-1-indanone (324 mg, 1 mmol), and the mixture was stirred for 30 minutes. Subsequently, sodium cyanoborohydride (126 mg) was added to the reaction solution, and the mixture was heated to reflux for 22 hours. The reaction liquid was allowed to be cooled to room temperature and then poured in water, extracted with dichloromethane, and dried with anhydrous sodium sulfate. The extracted liquid was concentrated under reduced pressure to obtain a light yellow solid of 11-{[1-(methylamino)indan-5-yl]oxy}-1-(3,6,9-trioxa) undecanol (369 mg). The obtained methylamine compound (369 mg) was dissolved in 1,2-dichloroethane (4 mL), 4-(dimethylamino)cinnamaldehyde (175 mg) and sodium triacetoxyborohydride (371 mg) were added thereto, and the mixture was stirred at room temperature for 21 hours. The reaction liquid was poured in a saturated sodium hydrogen carbonate aqueous solution, extracted with dichloromethane, and dried with anhydrous sodium sulfate. The residue obtained by concentrating the extracted liquid under reduced pressure was dissolved in methanol (5 mL), Pd/Fib (2.5%, 50 mg) was added thereto, and the mixture was allowed to react at room temperature for 22 hours under a hydrogen atmosphere at 1 atm (balloon). The reaction mixture was filtrated, the residue obtained by concentrating the filtrate under reduced pressure was purified with column chromatography (silica gel, dichloromethane/methanol=9/1), and a light yellow oily compound as titled above (240 mg, 48%) was yielded.

¹H NMR (400 MHz, CDCl₃) δ: 1.72-1.85 (m, 2H, CH₂CH₂CH₂), 1.99-2.06 (m, 2H, ArCH₂CH₂CH) 2.16 (s, 3H, NCH₃), 2.38-2.63 (complex, 4H, ArCH₂ and NCH₂), 2.7-2.8 (m, 1H, ArCH₂), 2.82-2.91 (m, 1H, ArCH₂), 2.9 (s, 6H, N(CH₃)₂), 3.61 (t, J=4.6 Hz, 2H, OCH₂), 3.66-3.76 (complex, 10H, OCH₂), 3.85 (t, J=4.8 Hz, 2H, OCH₂), 4.12 (t, J=5 Hz, 2H, ArOCH₂), 4.36 (br t, J=6.9 Hz, 1H, ArCHN), 6.69 (d, J=8.7 Hz, 2H, ArH), 6.76 (s, 1H, ArH), 6.76-6.79 (1H, ArH), 7.06 (d, J=8.7 Hz, 2H, ArH), 7.23 (d, J=8.2 Hz, 1H, ArH)

Compound 3 (C3, Hereafter, which May be Referred to as “GIF-2282”):

Compound 3 (8 mg, 22%) was yielded from 11-{[1-({3-[4-(dimethylamino)phenyl]propyl}(methyl)amino)indan-5-yl]oxy}-1-(3,6,9-trioxa)undecanol (27.5 mg, 55 mol) by the same scheme as in the synthesis of Compound 2.

¹H NMR (400 MHz, CDCl₃) δ: 1.8-1.9 (m, 2H, CH₂CH₂CH₂), 2.05-2.15 (m, 2H, ArCH₂CH₂CH), 2.22 (s, 3H, NCH₃), 2.46-2.9 (complex, 6H, ArCH₂ and NCH₂), 2.90 (s, 6H, N(CH₃)₂), 3.65-3.78 (complex, 8H, OCH₂), 3.82-3.9 (m, 2H, OCH₂), 4.02 (t, J=4.6 Hz, 2H, OCH₂), 4.08-4.14 (m, 2H, OCH₂), 4.45-4.5 (br, 1H, ArCHN), 4.55 (t, J=4.6 Hz, 2H, ArOCH₂), 6.67 (dd, J=3.4 and 8.5 Hz, 2H, ArH), 6.7-6.8 (complex, 3H, ArH), 7.22-7.26 (1H, ArH), 8.50 (d, J=8.7 Hz, 1H, ArH)

### Example 4

Compound 4 (C4) was synthesized in the procedure described below.

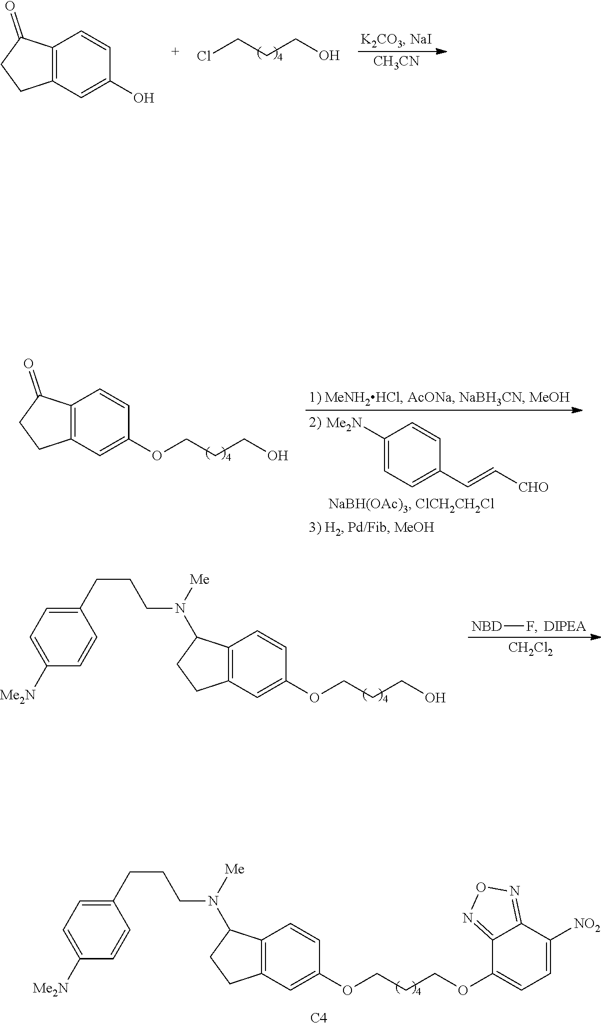

### 5-((6-hydroxyhexyl)oxy)-1-indanone

Potassium carbonate (415 mg), 6-chloro-1-hexanol (0.8 mL), and sodium iodide (30 mg) were added to acetonitrile (10 mL) solution containing 5-hydroxy-1-indanone (296 mg, 2 mmol), and the mixture was heated to reflux for 30 hours. The reaction liquid was allowed to be cooled to room temperature and then poured in water, and the resulting product was extracted with ethyl acetate, washed with sodium thiosulfate aqueous solution and a saline solution, and dried with anhydrous sodium sulfate. The residue obtained by concentrating the extracted liquid under reduced pressure was purified with column chromatography (silica gel, ethyl acetate/hexane=2/1), and a white solid of the compound as titled above (424 mg, 85%) was yielded.

¹H NMR (400 MHz, CDCl₃) δ: 1.41-1.67 (complex, 8H, CH₂), 1.84 (quin, J=6.9 Hz, 2H, CH₂CH₂CH₂) 2.67 (t J=5.9 Hz, 2H, COCH₂), 3.08 (t J=5.9 Hz, 2, ArCH₂), 3.62-3.72 (m, 2H, CH₂OH), 4.04 (t, J=6.4 Hz, 2H, ArOCH₂), 6.85-6.92 (br, 2H, ArH), 7.68 (d, J=9.2 Hz, 1H, ArH)

### 6-{[1-({3-[4-(dimethylamino)phenyl]propyl}(methyl)amino)indan-5-yl]oxy}-1-hexanol

The compound as titled above (421 mg, 59% in three steps) was yielded by using 5-((6-hydroxyhexyl)oxy)-1-indanone (419 mg, 1.69 mmol) by the same scheme as in the synthesis of intermediate dialkylamine of Compound 3.

¹H NMR (400 MHz, CDCl₃) δ: 1.38-1.54 (complex, 4H, CH₂), 1.60 (quin, J=6.9 Hz, 2H, CH₂CH₂CH₂), 1.74-1.83 (complex, 4H, CH₂), 1.99-2.06 (m, 2H, CH₂), 2.16 (s, 3H, NCH₃), 2.37-2.63 (complex, 4H, ArCH₂ and NCH₂), 2.71-2.8 (m, 1H, ArCH₂), 2.82-2.9 (m, 1H, ArCH₂), 2.9 (s, 6H, N(CH₃O), 3.65 (t, J=6.7 Hz, CH₂OH), 3.94 (t, J=6.4 Hz, 2H, ArOCH₂), 4.36 (t J=6.9 Hz, 1H, ArCHN), 6.69 (d, J=8.7 Hz, 2H, ArH), 6.73 (s, 1H, ArH), 6.73-6.76 (1H, ArH), 7.06 (d, J=8.7 Hz, 2H), 7.23 (d, J=8.2 Hz, ArH)

Compound 4 (C4, Hereafter, which May be Referred to as “GIF-2280”):

Compound 4 (30 mg, 93%) was yielded from 6-{[1-({3-[4-(dimethylamino)phenyl]propyl}(methyl)amino)indan-5-yl]oxy}-1-hexanol (23 mg, 55 mol) by the same scheme as in the synthesis of Compound 2.

¹H NMR (400 MHz, CDCl₃) δ: 1.57-1.72 (complex, 8H, CH₂), 1.79-1.87 (m, 2H, CH₂), 2.0-2.08 (m, 2H, CH₂), 2.17 (s, 3H, NCH₃), 2.44-2.63 (complex, 4H, ArCH₂ and NCH₂), 2.72-2.9 (complex, 2H, ArCH₂), 2.9 (s, 6H, N(CH₃) 2), 3.97 (t, J=6.4 Hz, 2H, ArOCH₂), 4.38-4.43 (2H, ArOCH₂ and ArCHN) 6.65 (d, J=8.5 Hz, 1H, ArH) 6.68 (d, J=8.7 Hz, 2H, ArH), 6.73 (s, 1H, ArH), 6.73-6.76 (1H, ArH), 7.06 (d, J=8.7 Hz, 2H, ArH), 8.53 (d, J=8.5 Hz, 1H, ArH)

### Example 5

Compound 5 (C5) was synthesized in the procedure described below.

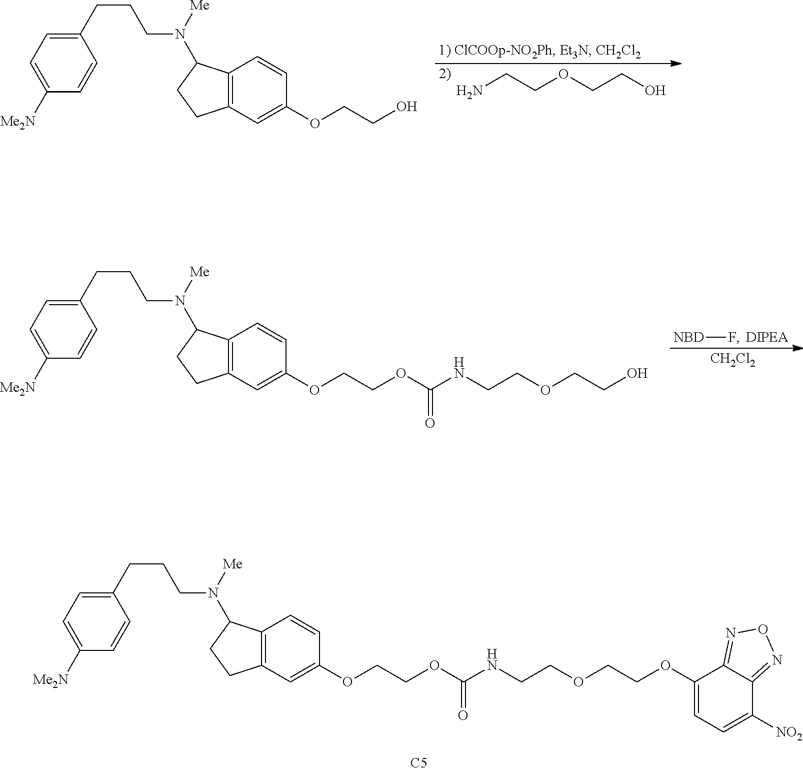

### 2-{[1-({3-[4-(dimethylamino)phenyl]propyl}(methyl)amino) indan-5-yl]oxy}ethyl [2-(2-hydroxyethoxy)ethyl]carbamate

2-{[1-({3-[4-(dimethylamino)phenyl]propyl}(methyl)amino) indan-5-yl]oxy}-1-ethanol (56 mg, 152 mol), which is a synthetic intermediate of Compound 1, was dissolved in dichloromethane (0.5 mL), triethylamine (64 μL) and 4-nitrophenyl chloroformate (37 mg) were sequentially added thereto at room temperature, and the mixture was allowed to react for 15 hours. Subsequently, 2-(2-aminoethoxy) ethanol (23 μL) was added at room temperature to the reaction liquid, and the mixture was stirred for another 7 hours. The reaction mixture was poured in a saturated sodium hydrogen carbonate aqueous solution, extracted with dichloromethane, and dried with anhydrous sodium sulfate. The residue obtained by concentrating the extracted liquid under reduced pressure was purified with column chromatography (amino silica gel, ethyl acetate), and the compound as titled above (26 mg, 34%) was yielded.

¹H NMR (400 MHz, CDCl₃) δ: 1.7-1.82 (m, 2H, CH₂CH₂CH₂), 2.02 (q, J=7.3 Hz, 2H, ArCH₂CH₂CH), 2.15 (s, 3H, NCH₃), 2.38-2.46 and 2.46-2.62 (complex, 4H, ArCH₂ and NCH₂), 2.7-2.79 and 2.8-2.9 (complex, 2H, ArCH₂), 2.90 (s, 6H, N(CH₃)₂), 3.34-3.42 (m, 2H, CONHCH₂), 3.56 (br m, 4H, OCH₂), 3.72 (t, J=4.6 Hz, 2H, OCH₂), 4.12-4.17 (br m, 2H, OCH₂), 4.35 (t, J=6.9 Hz, 1H, ArCHN), 4.39-4.44 (br, 2H, ArOCH₂), 5.38 (br s, 1H, CONH), 6.69 (d, J=8.6 Hz, 2H, ArH), 6.74 (s, 1H, ArH), 6.74-6.78 (m, 1H, ArH), 7.06 (d, J=8.6 Hz), 7.24 (d, J=7.8 Hz, 1H, ArH)

Compound 5 (C5, Hereafter, which May be Referred to as “GIF-2279”):

Compound 5 (7.1 mg, 21%) was yielded from carbamate described above (26 mg, 52 mol) by the same scheme as in the synthesis of Compound 2.

¹H NMR (400 MHz, CDCl₃) δ: 1.75-1.87 (m, 2H, CH₂CH₂CH₂), 2.0-2.1 (2H, ArCH₂CH₂CH), 2.18 (s, 3H, NCH₃), 2.42-2.63 (complex, 4H, ArCH₂ and NCH₂), 2.71-2.9 (complex, 2H, ArCH₂), 2.90 (s 6H, N(CH₃) 2), 3.38-3.46 (m, 2H, CONHCH₂), 3.64-3.68 (m, 2H, OCH₂), 3.96-4.0 (br, 2H, OCH₂), 4.1-4.16 (2H, OCH₂), 4.37-4.44 (br, 3H, OCH₂ and ArCHN), 4.54 (br t, J=4.1 Hz, 2H, ArOCH₂), 5.15 (br s, CONH), 6.68 (d, J=8.7 Hz, 2H, ArH), 6.73 (s, 1H, ArH), 6.73-6.77 (m, 1H, ArH), 7.05 (d, J=8.7 Hz, 2H, ArH), 7.23-7.26 (1H, ArH), 8.51 (d, J=8.2 Hz, 1H, ArH)

### Example 6

Compound 6 (C6) was synthesized in the procedure described below.

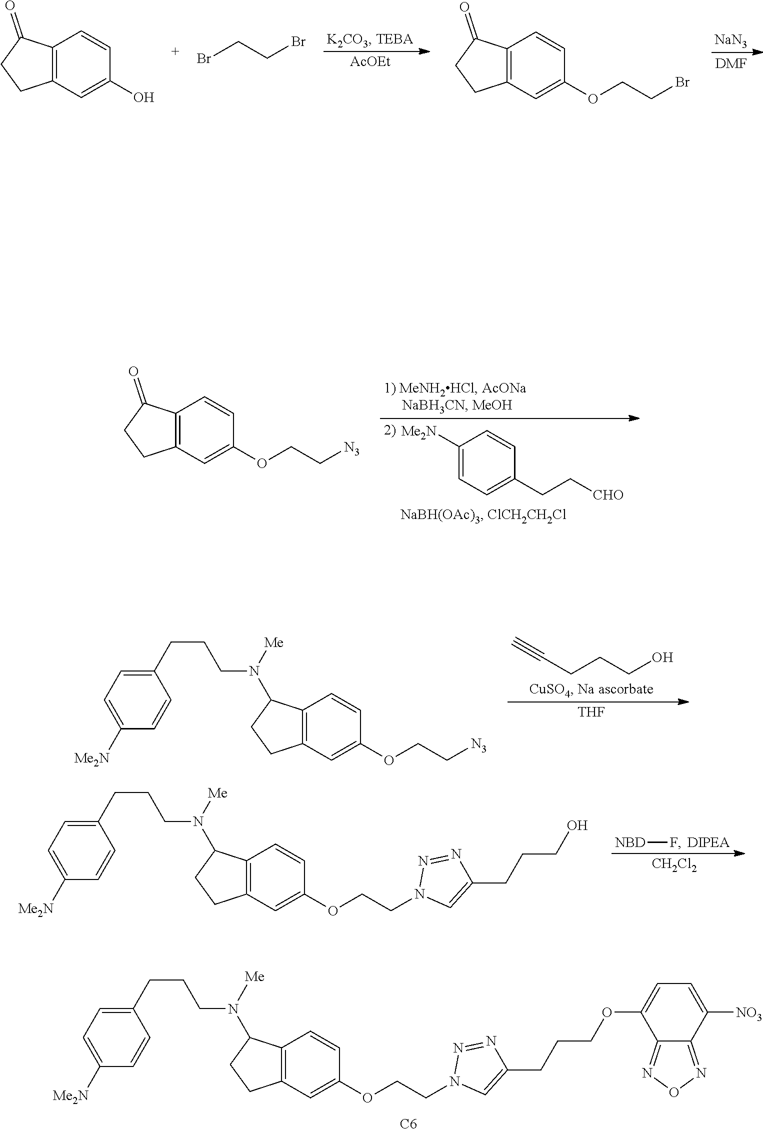

### 5-(2-bromoethoxy)-1-indanone

After 5-hydroxy-1-indanone (745 mg, 5.03 mmol) was dissolved in ethyl acetate (15 mL), potassium carbonate (2.45 g) suspended in ethyl acetate (15 mL), 1,2-dibromoethane (3.27 mL), and benzyltriethyl ammonium chloride (121 mg) were then sequentially added thereto, and the mixture was heated to reflux to 24 hours. The reaction liquid was allowed to be cooled to room temperature and then poured in ice water, extracted with ethyl acetate, washed with a saturated saline solution, and dried with anhydrous sodium sulfate. The residue obtained by concentrating the extracted liquid under reduced pressure was purified with column chromatography (silica gel, hexane/ethyl acetate=1/1), and a white solid of the compound as titled above (1.06 g, 83%) was yielded.

¹H NMR (400 MHz, CDCl₃) δ: 2.67 (m, 2H, COCH₂), 3.08 (br t, J=6 Hz, 2H, ArCH₂), 3.66 (t, J=6.4 Hz 2H, BrCH₂), 4.35 (t, J=6.4 Hz, 2H, OCH₂), 6.89-6.93 (br m, 2H, ArH), 7.69 (d, J=9.2 Hz, 1H, ArH)

### 5-(2-azidoethoxy)-1-indanone

5-(2-bromoethoxy)-1-indanone (410 mg, 1.61 mmol) was dissolved in N,N-dimethylformamide (5.0 mL), sodium azide (129 mg) was added thereto, and the mixture was heated to reflux for 35 minutes. The reaction liquid was allowed to be cooled to room temperature and then poured in water, extracted with diethyl ether, washed with a saturated saline solution, and dried with anhydrous sodium sulfate. The extracted liquid was concentrated under reduced pressure, and a reddish brown solid of the compound as titled above (346 mg, 99%) was yielded.

¹H NMR (400 MHz, CDCl₃) δ: 2.66-2.7 (m, 2H, COCH₂), 3.09 (br t, J=6 Hz, 2H, ArCH₂), 3.63 (t, J=5 Hz, 2H, N₃CH₂), 4.21 (t, J=5 Hz, 2H, OCH₂), 6.90-6.94 (br m, 2H, ArH), 7.70 (d, J=9.2 Hz, 1H, ArH)

### 5-(2-azidoethoxy)-N-{3-[4-(dimethylamino)phenyl]propyl}-N-methyl-1-indanylamine

Methylamine hydrochloride (101 mg) and sodium acetate (123 mg) were added at room temperature to methanol (2 mL) solution containing 5-(2-azidoethoxy)-1-indanone (109 mg, 0.5 mmol), and the mixture was stirred for 30 minutes. Subsequently, sodium cyanoborohydride (63 mg) was added to the reaction solution, and the mixture was heated to reflux for 30 minutes. The reaction liquid was allowed to be cooled to room temperature and then poured in water, extracted with dichloromethane, and dried with anhydrous sodium sulfate. The extracted liquid was concentrated under reduced pressure to obtain light brown oily 5-(2-azidoethoxy)-N-methyl-1-indanylamine (118 mg). The obtained methylamine compound (118 mg) was dissolved in 1,2-dichloroethane (2 mL), 3-[4-(dimethylamino)phenyl]propanal (88 mg) and sodium triacetoxyborohydride (185 mg) were added thereto, and the mixture was stirred at room temperature for 23 hours. The reaction liquid was poured in a saturated sodium hydrogen carbonate aqueous solution, extracted with ethyl acetate, and dried with anhydrous sodium sulfate. The residue obtained by concentrating the extracted liquid under reduced pressure was purified with column chromatography (silica gel, acetone/ethyl acetate=1/1), and a light yellow oily compound as titled above (161 mg, 82%) was yielded.

¹H NMR (400 MHz, CDCl₃) δ: 1.72-1.85 (m, 2H, CH₂CH₂CH₂), 2.03 (br q, J=7.6 Hz, 2H, ArCH₂CH₂CH), 2.16 (s, 3H, NCH₃), 2.38-2.64 (complex, 4H, ArCH₂ and NCH₂), 2.72-2.9 (complex, 2H, ArCH₂), 2.90 (s, 6H, N(CH₃)₂), 3.58 (t, J=4.6 Hz, 2H, CH₂N₃), 4.14 (t, J=4.6 Hz, 2H, ArOCH₂), 4.36 (t, J=6.9 Hz, 1H, ArCHN), 6.69 (d, J=8.2 Hz, 2H, ArH), 6.76 (s, 1H, ArH), 6.76-6.78 (1H, ArH), 7.06 (d, J=8.2 Hz, 2H, ArH), 7.23-7.26 (1H, ArH)

### 3-[1-(2-{[1-({3-[4-(dimethylamino)phenyl]propyl}(methyl)amino)indan-5-yl]oxy}ethyl)-1H-1,2,3-triazol-4-yl]-1-propanol

4-pentin-1-ol (7.8 μL), water (50 μL), copper sulfate aqueous solution (0.16 M, 50 μL), and sodium ascorbate (4.8 mg) were sequentially added to THE (300 μL) solution containing 5-(2-azidoethoxy)-N-{3-[4-(dimethylamino)phenyl]propyl}-N-methyl-1-indanylamine (30 mg, 76 μL), and the mixture was stirred at room temperature for 2 hours. The reaction mixture was poured in a saturated sodium hydrogen carbonate aqueous solution, extracted with dichloromethane, and dried with anhydrous sodium sulfate. The residue obtained by concentrating the extracted liquid under reduced pressure was purified with column chromatography (silica gel, acetone/ethyl acetate/methanol=4/4/1.5), and a light yellow oily compound as titled above (36 mg, quantitative) was yielded.

¹H NMR (400 MHz, CDCl₃) δ: 1.72-1.84 (m, 2H, CH₂CH₂CH₂), 1.93 (quin, J=6.8 Hz, 2H, CH₂CH₂CH₂) 1.99-2.06 (m, 2H, ArCH₂CH₂CH), 2.15 (s, 3H, NCH₃), 2.38-2.62 (complex, 4H, ArCH₂ and NCH₂) 2.7-2.9 (complex, 2H, ArCH₂), 2.83 (t, J=7.3 Hz, 2H, C═CCH₂), 2.9 (s, 6H, N(CH₃)₂), 3.69 (t, J=6.2 Hz, 2H, CH₂H), 4.32 (t, J=5 Hz, 2H, OCH₂N), 4.35 (t, J=6.9 Hz, 1H, ArCHN), 4.71 (t, J=5 Hz 2H, ArOCH₂CH₂N), 6.67-6.73 (complex, 4H, ArH), 7.06 (d, J=8.7 Hz, 2H, ArH), 7.24 (d, J=7.8 Hz, 1H, ArH), 7.52 (s, 1H, NCH═C)

Compound 6 (C6, Hereafter, which May be Referred to as “GIF-2278”):

Compound 6 (21 mg, 60%) was yielded from triazole compound described above (26 mg, 55 mol) by the same scheme as in the synthesis of Compound 2.

¹H NMR (400 MHz, CDCl₃) δ: 1.74-1.84 (m, 2H, CH₂CH₂CH₂), 1.88-1.95 (m, 1H, CH₂CH₂CH₂) 1.98-2.08 (m, 3H, CH₂CH₂CH₂ and ArCH₂CH₂CH), 2.18 (s, 3H, NCH₃), 2.36-2.62 (complex, 4H, ArCH₂ and NCH₂), 2.69-2.9 (complex, 2H, ArCH₂), 2.90 (s, 6H, N(CH₃) 2), 3.00 (t, J=7.1 Hz, 2H, C═CCH₂), 4.32 (t, J=5 Hz, 2H, ArOCH₂CH₂N), 4.35-4.44 (br, 1H, ArCHN), 4.48 (t, J=4.3 Hz, 2H, ArOCH₂), 4.73 (t, J=5 Hz, 2H, AOCH₂CH₂N), 6.62-6.72 (complex, 5H, ArH), 7.04 (d, J=8.7 Hz, 2H, ArH), 7.22-7.26 (1H, ArH), 7.59 (s, 1H, NCH═C), 8.49 (d, J=8.3 Hz, 1H, ArH)

### Example 7

Compound 7 (C7) was synthesized in the procedure described below.

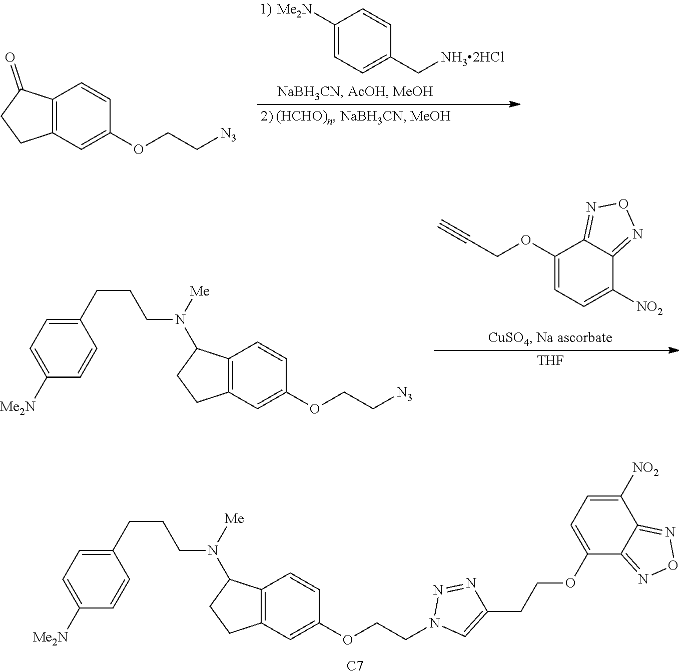

### 5-(2-azidoethoxy)-N-[4-(dimethylamino)benzyl]-1-indanylamine

4-(dimethylamino)benzylamine dihydrochloride (155 mg), triethylamine (216 μL), sodium cyanoborohydride (48.5 mg), and acetic acid (100 μL) were sequentially added to methanol (3 mL) solution containing 5-(2-azidoethoxy)-1-indanone (100 mg, 0.46 mmol), and the mixture was heated to reflux for 42 hours. The reaction liquid was allowed to be cooled to room temperature, and hydrochloric acid (1M, 2 mL) was then added thereto to quench excessive hydride. Subsequently, a sodium hydroxide aqueous solution (1M) was dropped into the reaction liquid until it became weakly basic, extraction was made with ethyl acetate, and the extract was dried with anhydrous sodium sulfate. The residue obtained by concentrating the extracted liquid under reduced pressure was purified with column chromatography (silica gel, ethyl acetate), and a light yellow oily compound as titled above (130 mg, 80%) was yielded.

¹H NMR (400 MHz, CDCl₃) δ: 1.88-2.0 and 2.35-2.45 (complex, each 1H, ArCH₂CH₂) 2.78 (dt, J=7.8 and 15.8 Hz, 1H, ArCH₂), 2.91 (s, 6H, N(CH₃)₂), 3.0 (ddd, J=5, 8.5, and 15.8, 1H ArCH₂), 3.56 (t, J=5 Hz, 2H, N₃CH₂), 3.77 and 3.81 (d, J=12.8 Hz, 2H, ArCH₂N), 4.11 (t, J=5 Hz, 2H OCH₂), 4.26 (t, J=6.4 Hz, 1H, ArCHN), 6.70 (d, J=8.7 Hz, 2H, ArH), 6.76 (d, J=8.3 Hz, 1H, ArH), 6.78 (br s, 1H, ArH), 7.22-7.25 (2H, ArH), 7.27 (d, J=8.3 Hz, 1H, ArH)

### 5-(2-azidoethoxy)-N-[4-(dimethylamino)benzyl]-N-methyl-1-indanylamine

5-(2-azidoethoxy)-N-[4-(dimethylamino)benzyl]-1-indanylamine (57.5 mg, 0.164 mmol) was dissolved in methanol (0.8 mL), paraformaldehyde (27.1 mg) and sodium cyanoborohydride (16.0 mg) were sequentially added thereto, and the mixture was allowed to react at room temperature for 7 hours. Hydrochloric acid (1M) was added to the reaction liquid to quench excessive hydride. Subsequently, a sodium hydroxide aqueous solution (1M) was dropped into the reaction liquid until it became weakly basic, extraction was made with ethyl acetate, and the extract was dried with anhydrous sodium sulfate. The residue obtained by concentrating the extracted liquid under reduced pressure was purified with column chromatography (silica gel, ethyl acetate), and a light yellow oily compound as titled above (58 mg, 97%) was yielded.

¹H NMR (400 MHz, CDCl₃) δ: 1.6-1.8 and 2.0-2.11 (complex, each 1H, ArCH₂CH₂), 2.12 (s, 3H, NCH₃), 2.71-2.83 and 2.86-2.95 (complex, each 1H, ArCH₂), 2.91 (s, 6H, N(CH₃)₂), 3.32 (d, J=12.8 Hz, 1H, ArCH₂), 3.50 (d, J=12.8 Hz, 1H, ArCH₂), 3.57 (t, J=5 Hz, 2H, NCH₂), 4.13 (t J=5 Hz, 2H, OCH₂), 4.40 (t, J=7.1 Hz, 1H, ArCHN), 6.70 (d, J=8.7 Hz, 2H, ArH), 6.76 (s, 1H, ArH), 6.78 (d, J=8.2 Hz, 1H, ArH), 7.21 (d, J=8.7 Hz, 2H, ArH), 7.33 (d, J=8.2 Hz, 1H, ArH)

Compound 7 (C7, Hereafter, which May be Referred to as “GIF-2274”):

3-butin-1-ol (23 μL) and N,N-diisopropylethylamine (42 μL) were added to dichloromethane (1 mL) solution containing NBD-F (36.6 mg, 0.2 mmol), and the mixture was allowed to react at room temperature for 20 hours. The reaction liquid was poured in a saturated saline solution, extracted with ethyl acetate, and dried with anhydrous sodium sulfate. The extracted liquid was concentrated under reduced pressure to obtain 4-(3-butin-1-yloxy)-7-nitrobenzofurazan (46.7 mg). This resulting crude product (10 mg) and 5-(2-azidoethoxy)-N-[4-(dimethylamino)benzyl]-N-methyl-1-indanylamine (17 mg) were dissolved in THE (0.1 mL), copper sulfate aqueous solution (0.08 M, 25 μL) and sodium ascorbate (2.2 mg) were added thereto, and the mixture was allowed to react at room temperature for 17 hours. Water was added to the reaction liquid, and extraction was made with ethyl acetate, and the extracted was dried with anhydrous sodium sulfate. The residue obtained by concentrating the extracted liquid under reduced pressure was purified with column chromatography (silica gel, acetone), and a black solid of Compound 7 (6.5 mg, 25%) was yielded.

¹H NMR (400 MHz, CDCl₃) δ: 2.07-2.2 (complex, 2H, ArCH₂CH₂CH), 2.14 (s, 3H, NCH₃), 2.71-2.82 (m, 1H, ArCH₂), 2.85-2.92 (1H, ArCH₂), 3.37-3.57 (complex, 4H, C═CCH₂ and ArCH₂N), 4.27-4.35 (complex, 2H, ArOCH₂CH₂N), 4.52 (t, J=6.9 Hz, 1H, ArCH₂N), 4.67 (t, J=6.4 Hz, 2H, ArOCH₂), 4.74 (t, J=4.6 Hz, 2H, ArOCH₂), 6.65-6.75 (complex, 5H, ArH), 7.20 (d, J=8.7 Hz, 2H, ArH), 7.32 (d, J=7.8 Hz, 1H, ArH), 7.79 (s, 1H, NCH═C), 8.48 (d, J=8.2 Hz, 1H ArH)

### Example 8

Compound 8 (C8) was synthesized in the procedure described below.

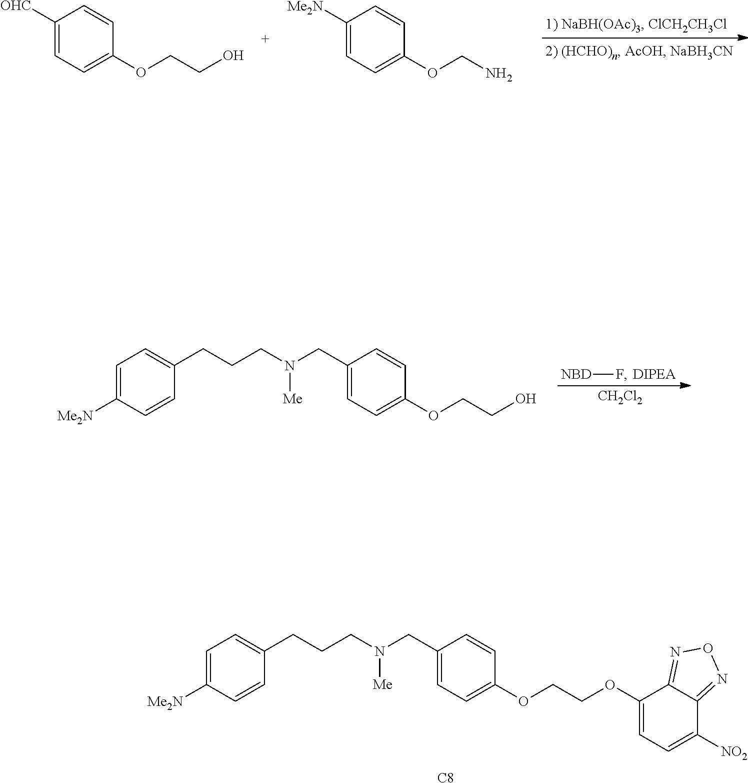

### 2-[4-({3-[4-(dimethylamino)phenyl]propyl}(methyl)amino)phenoxy]-1-ethanol

4-(2-aminoethyl)N,N-dimethylaniline (196 mg) dissolved in 1,2-dichloroethane (1 mL) was added to 1,2-dichloroethane solution (3 mL) containing 4-(2-hydroxyethoxy)benzaldehyde (166 mg, 1 mmol), and the mixture was stirred at room temperature for 1 hour. Sodium triacetoxyborohydride (371 mg) was added to this solution, and the mixture was stirred at room temperature for 15 hours. The reaction liquid was poured in a saturated sodium hydrogen carbonate aqueous solution, extracted with dichloromethane, and dried with anhydrous sodium sulfate. The residue (416 mg) obtained by concentrating the extracted liquid under reduced pressure was dissolved in methanol (4 mL), paraformaldehyde (90 mg), acetic acid (57 μL), and sodium cyanoborohydride (94 mg) were added thereto, and the mixture was allowed to react at room temperature for 20 hours. The reaction liquid was poured in a saturated sodium hydrogen carbonate aqueous solution, extracted with ethyl acetate, washed with a saturated saline solution, and dried with anhydrous sodium sulfate. The residue obtained by concentrating the extracted liquid under reduced pressure was purified with column chromatography (silica gel, dichloromethane/methanol=9/1), and a light yellow oily compound as titled above (232 mg, 68%) was yielded.

¹H NMR (400 MHz, CDCl₃) δ: 1.80 (quin, J=7.6 Hz, 2H, CH₂CH₂CH₂), 2.17 (s, 3H, NCH₃), 2.39 (t, J=7.6 Hz, 2H, NCH₂CH₂), 2.54 (t, J=7.6 Hz, 2H, ArCH₂CH₂), 2.91 (s, 6H, N(CH₃) 2), 3.42 (s, 2H, ArCH₂N), 3.95 (t, J=4.5 Hz, 2H, CH₂OH), 4.08 (t, J=4.5 Hz, 2H, ArOCH₂), 6.69 (d, J=8.5 Hz, 2H, ArH) 6.86 (d, J=8.5 Hz, 2H, ArH), 7.05 (d, J=8.5 Hz, 2H, ArH), 7.22 (d, J=8.5 Hz, 2H, ArH)

Compound 8 (C8, Hereafter, which May be Referred to as “GIF-2281”):

Compound 8 (15 mg, 54%) was yielded from 2-[4-({3-[4-(dimethylamino)phenyl]propyl}(methyl)amino)phenoxy]-1-ethanol described above (19 mg, 55 mol) by the same scheme as in the synthesis of Compound 2.

¹H NMR (400 MHz, CDCl₃) δ: 1.79-1.88 (m, 2H, CH₂CH₂CH₂), 2.29 (s, 3H, NCH₃) 2.49-2.55 (complex, 4H, ArCH₂CH₂CH₂N), 3.54-3.58 (br, 2H, ArCH₂N), 4.49 (t, J=4.7 Hz, 2H, ArOCH₂CH₂), 4.78 (t, J=4.7 Hz, 2H, ArOCH₂CH₂) 6.68 (d, J=8.7 Hz, 2H, ArH), 6.79 (d, J=Hz, 1H, ArH), 6.87 (d, J=8.7 Hz, 2H, ArH), 7.04 (d, J=8.7 Hz, 2H, ArH), 7.23 (d, J=8.7 Hz, 2H, ArH), 8.56 (d J=8.7 Hz, 1H, ArH)

### Example 9

Compound 9 (Hereafter, which May be Referred to as “GIF-2283”):

Compound 9 (16.4 mg, 56%) was yielded from 2-(4-{6-[4-(dimethylamino)phenyl]-3-methyl-3-azahexyl}phenoxy)-1-ethanol (20 mg, 55 mol) by the same scheme as in the synthesis of Compound 2.

¹H NMR (400 MHz, CDCl3) δ: 1.77-1.86 (br, 2H, ArCH₂CH₂CH₂), 2.38 (s, 3H, NCH₃) 2.5-2.57 (complex, 4H, ArCH₂), 2.63-2.8 (complex, 4H, NCH₂), 2.90 (s, 6H, N(CH₃)₂), 3.94 (t, J=4.5 Hz, 2H, ArOCH₂), 4.05 (t, J=4.5 Hz, 2H, ArOCH₂), 6.68 (d, J=8.7 Hz, 2H, ArH), 6.79 (d, J=8.5 Hz, 1H, ArH), 6.84 (d, J=8.7 Hz, 2H, ArH), 7.04 (d, J=8.7 Hz, 2H, ArH), 7.12 (d, J=8.7 Hz, 2H, ArH), 8.55 (d, J=8.5 Hz, 1H, ArH)

### [Staining and Detection of Extracellular Vesicles Using Synthesized Compound]

### Example 10 and Comparative Example 1: Staining Effect of Each Synthesized Compound

### Preparation of Extracellular Vesicles (Exosomes)

Commercially available pasteurized milk (by Seki milk or Takanashi) of 1 L was centrifuged at 8000 rpm for 30 minutes, and the supernatant was filtrated by a coffee filter. Subsequently, acetic acid was added thereto to be 1%, and the mixture was further centrifuged at 8000 rpm for 30 minutes. The supernatant thereof was further filtrated by a coffee filter. The liquid obtained after the filtration was filtrated by a filter of 0.45 μm (by Sartorius), and the filtrate was filtrated by a filter of 0.1 μm (by Sartorius). Since extracellular vesicles remain on the filter of 0.1 μm, the extracellular vesicles were washed with 5 mL of phosphate buffer solution three times. Eventually, extracellular vesicles were collected with 1 mL of phosphate buffer solution. Further, 10 μL of the purified extracellular vesicle liquid was diluted with 40 μL of phosphate buffer solution (hereafter, referred to as “extracellular vesicle preparation liquid”)

### Production of the Staining Agent Used in Example 10

Compounds 1 to 9 synthesized in Examples 1 to 9 were diluted with DMSO to produce a staining agent used in Example 10.

### Production of the Staining Agent Used in Comparative Example 1

ExoSparkler Exosome Membrane Labeling Kit-Red (ExoSP Model No. EX02, by Dojin) was diluted with a DMSO solution in accordance with the instructions for use to produce a staining agent of Comparative example 1.

### Staining and Detection of Extracellular Vesicles

The staining agent produced in Example 10 was added to an extracellular vesicle preparation liquid so that the final concentration of the staining agent became 1 μM. Further, 1 μL of the staining agent produced in Comparative example 1 was added to an extracellular vesicle preparation liquid. In Example 10 and Comparative example 1, the staining agent was added to the extracellular vesicle preparation liquid and then allowed to stand at room temperature for 12 hours. After 12 hours elapsed, 10 μL of the reaction product was analyzed by a high-performance liquid chromatography gel filtration system. As the eluent for the column, 10 mM of Tris (pH 6.8), 0.25 mM of EDTA, and 50 mM of NaCl were used. Detection was performed on ExoSP at 560 nm for excitation and 600 nm for fluorescence detection and was performed on the compounds synthesized in Examples 1 to 8 at 480 nm for excitation and 530 nm for fluorescence detection. A column having 3 cm of Gen 2 qEV original/70 nm (by iZQN) and Shodex OHpak SB-807 (by Showa denko) connected to each other was used.

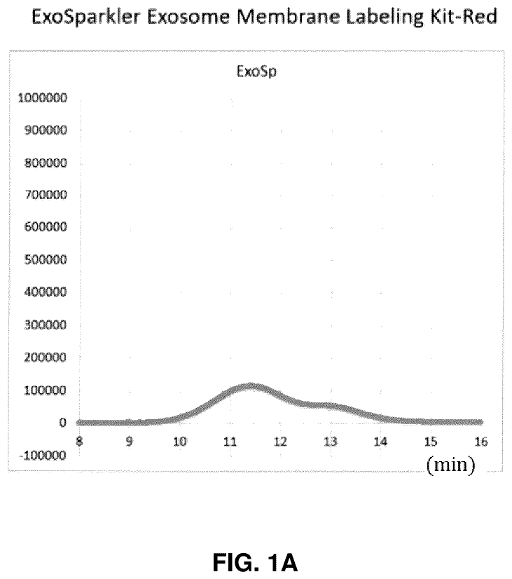

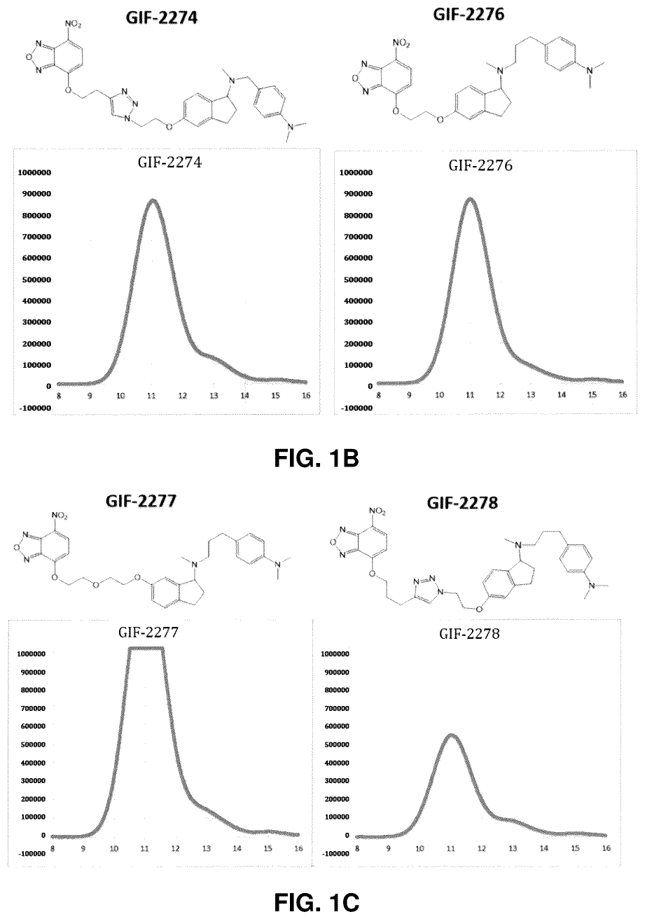

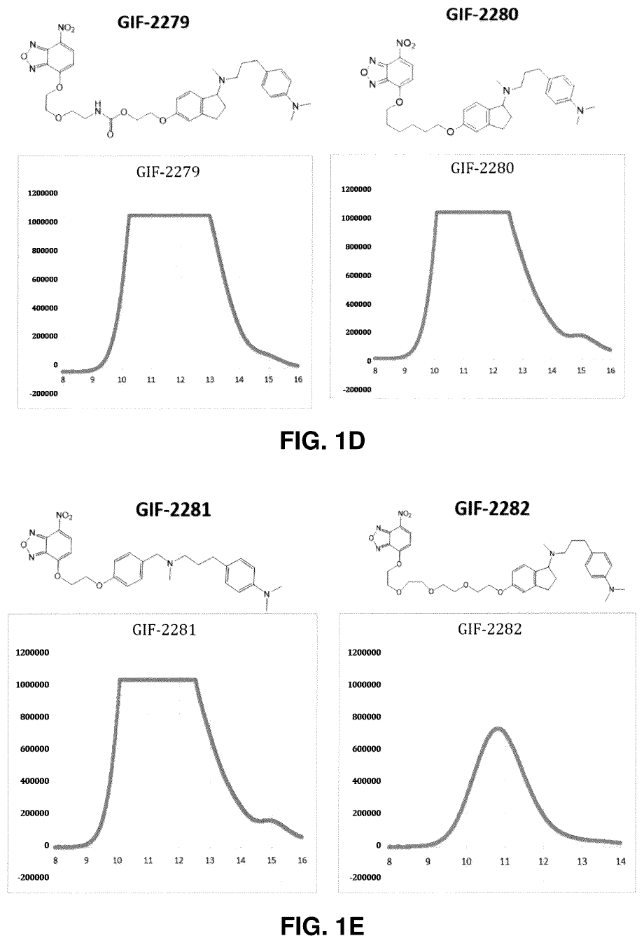

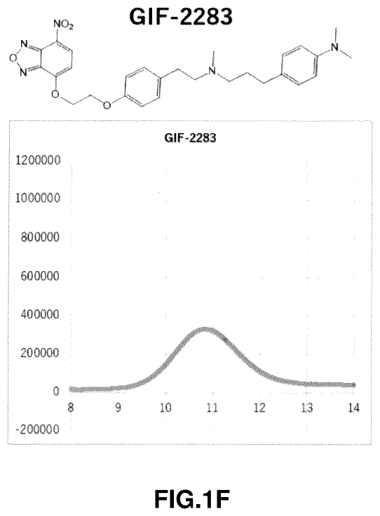

FIG. 1a to FIG. 1f illustrate the results. In the graphs, the horizontal axis represents the efflux time (minute), and the vertical axis represents the fluorescence intensity. In the graphs, respective numbers and chemical formulas of the synthesized compounds are also indicated. As is clear from the graphs illustrated in FIG. 1a to FIG. 1f, the compounds synthesized in Examples 1 to 9 exhibit a stronger fluorescence intensity than ExoSP of Comparative example 1 known as a staining agent for gel filtration of extracellular vesicles. Therefore, it was confirmed that the compounds disclosed in the present application can be used as a staining agent for gel filtration of extracellular vesicles.

### Example 11 and Comparative Example 2: Reaction Conditions

Next, experiments with different reaction conditions of the staining agent and the extracellular vesicle preparation liquid were performed.

### Example 11

Experiments were performed in the same procedure as in Example 10 except that, after the staining agent was added to the extracellular vesicle preparation liquid in Example 10, the mixture was immediately analyzed with a column (untreated), the mixture was analyzed after treated at 80° C. for 5 minutes, or the mixture was analyzed after treated at 80° C. for 15 minutes. Note that the compounds used in the experiments of Example 11 are labeled with the GIF numbers in the graphs.

### Comparative Example 2

Experiments were performed in the same procedure as in Comparative example 1 except that, after the staining agent was added to the extracellular vesicle preparation liquid in Comparative example 1, the mixture was immediately analyzed with a column (untreated), the mixture was analyzed after treated at 80° C. for 5 minutes, or the mixture was analyzed after treated at 80° C. for 15 minutes.

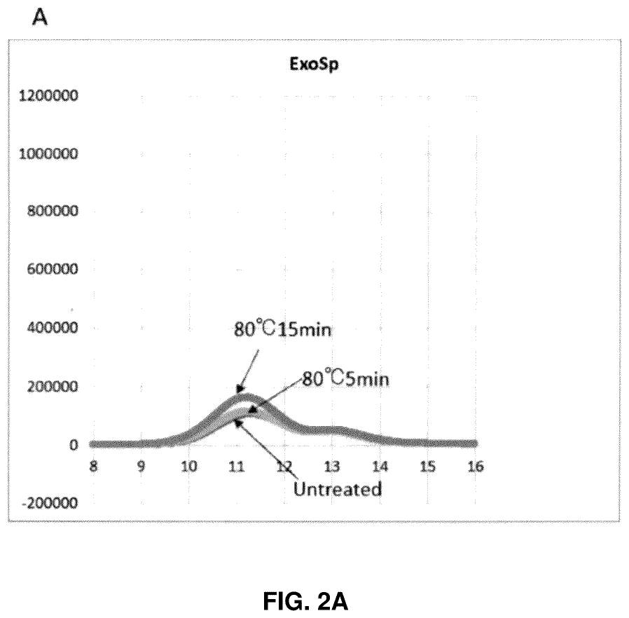

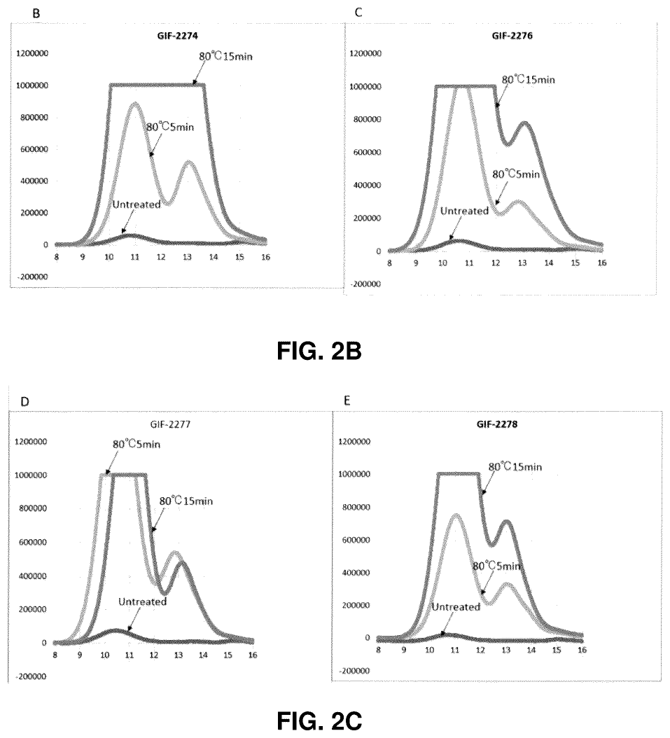

FIG. 2a to FIG. 2c illustrate the results. In the graphs, the horizontal axis represents the efflux time (minute), and the vertical axis represents the fluorescence intensity. As is clear from the graphs illustrated in FIG. 2a to FIG. 2c, ExoSP of Comparative example 1 exhibits little effect of heat treatment. In contrast, the compounds synthesized in Examples (GIF-2274, GIF-2276, GIF-2277, GIF-2278) all exhibit a significant increase in the fluorescence intensity due to heat treatment. A conceivable reason for this is an increase in the reaction rate. From the above results, it was confirmed that the compounds disclosed in the present application exhibit a significant increase in the binding of the staining agent to extracellular vesicles due to heating and thus are useful in short-time analysis.

### Example 12: Various Lactic Fermenting Beverages

Next, experiments using various lactic fermenting beverages were performed. As the lactic fermenting beverage, A: Yakult (by Yakult), B: Onakayorokobu lactic acid bacteria (by Kurashimoa), and C: Pirkle (by Nissin Foods) were used. Each 500 mL of the lactic fermenting beverages of A to C listed above was centrifuged at 8000 rpm for 30 minutes, and the supernatant was filtrated by a coffee filter. The liquid obtained after the filtration was filtrated by a filter of 0.45 μm (by Sartorius), and the filtrate was filtrated by a filter of 0.033 μm (by GVS). Since extracellular vesicles remain on the filter of 0.033 μm, the extracellular vesicles were washed with 5 mL of phosphate buffer solution three times. Eventually, extracellular vesicles were collected with 1 mL of phosphate buffer solution. The experiment was performed in the same procedure as in Example 11 except that GIF-2276 synthesized in Example 1 was added to 50 μL of the collected extracellular vesicle liquid so that the final concentration was 1 μM and respective samples were allowed to stand at 80° C. for 15 minutes and at room temperature for 12 hours.

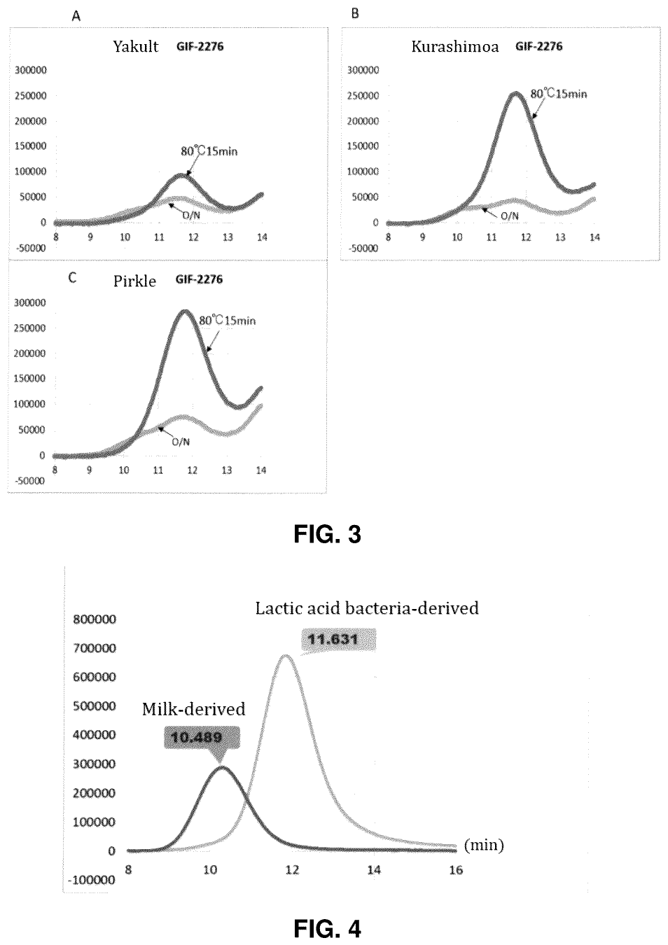

FIG. 3 illustrates the results. In the graph, the horizontal axis represents the efflux time (minute), and the vertical axis represents the fluorescence intensity. Further, “0/N” in the graph indicates a sample allowed to stand at room temperature for 12 hours. As is clear from FIG. 3, it was confirmed that the staining agents disclosed in the present application are useful for extracellular vesicles contained in the various lactic fermenting beverages. Further, it was also confirmed that the fluorescence intensity is significantly increased by heat treatment for extracellular vesicles contained in the lactic fermenting beverages in the same manner as in Example 11.

### Example 13: Size Separation of Extracellular Vesicles

Next, size separation of extracellular vesicles was performed by using GIF-2276 synthesized in Example 1 as the staining agent and using the milk-derived extracellular vesicles prepared in the procedure described in Example 10 and the lactic fermenting beverage (Yakult)-derived extracellular vesicles prepared in the procedure described in Example 12 as the samples. GIF-2276 was added to the extracellular vesicle preparation liquid so that the final concentration became 1 μM, the mixture was allowed to stand at room temperature for 12 hours, and the experiment was then performed in the procedure described in Example 10. FIG. 4 illustrates the result. As illustrated in FIG. 4, the milk-derived extracellular vesicles were eluted earlier than the lactic acid bacteria-derived extracellular vesicles. That is, this means that the milk-derived extracellular vesicles are larger than the lactic acid bacteria-derived extracellular vesicles. This was consistent with the fact that the milk-derived extracellular vesicles do not pass through the 0.1 μm filter, but the lactic acid bacteria-derived extracellular vesicles pass through the 0.1 μm filter. From the above results, it was confirmed that the use of the compounds (staining agents) disclosed in the present application enables size analysis of stained extracellular vesicles by a gel filtration system.

### Example 14 and Comparative Example 3: Staining Effect of Lactic Acid Bacteria Extracellular Vesicles of Each Synthesized Compound

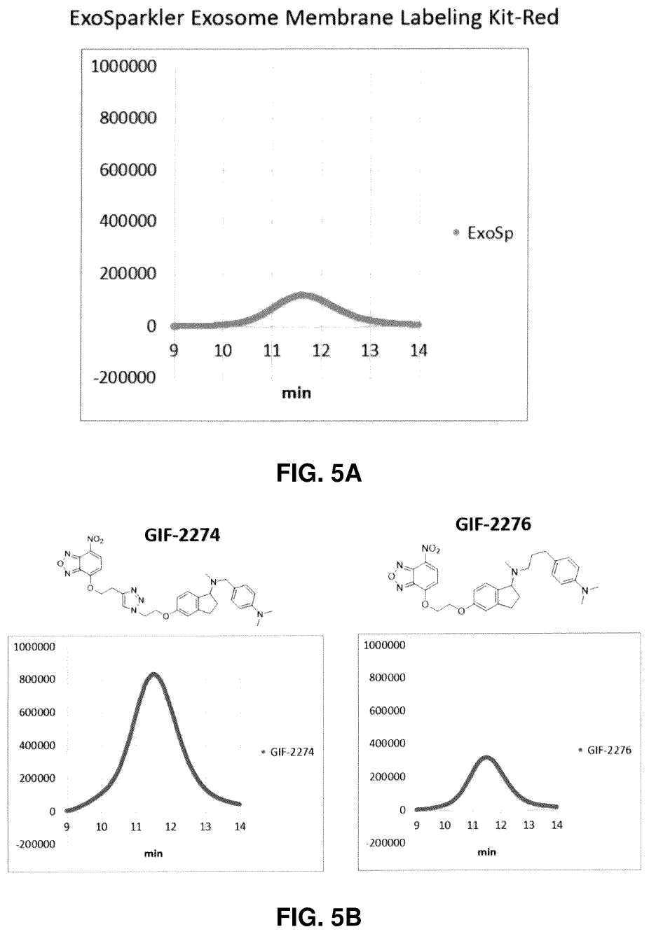

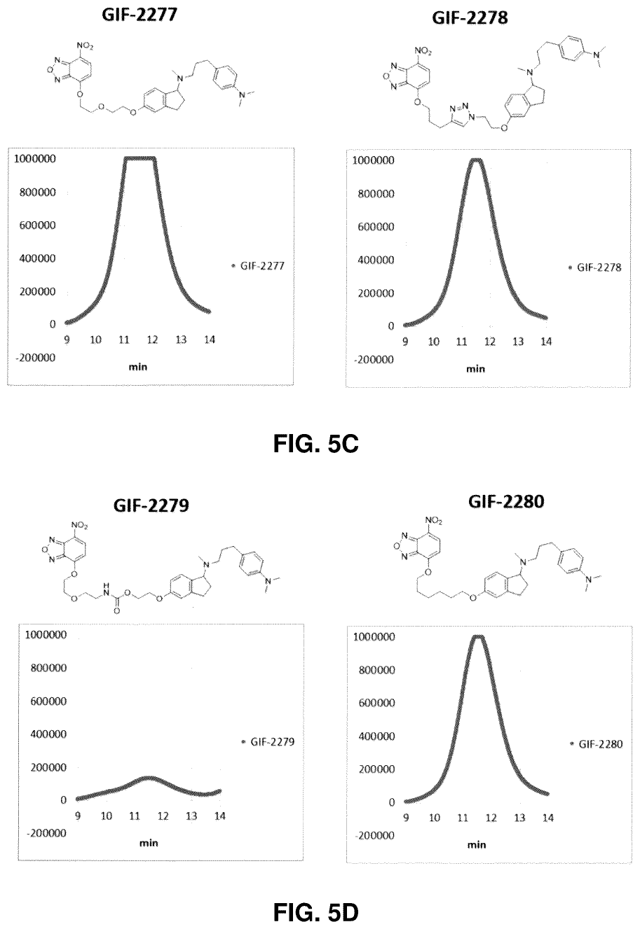

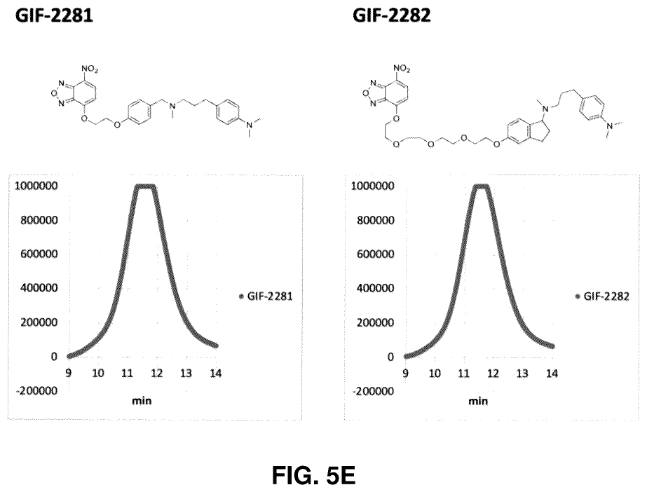

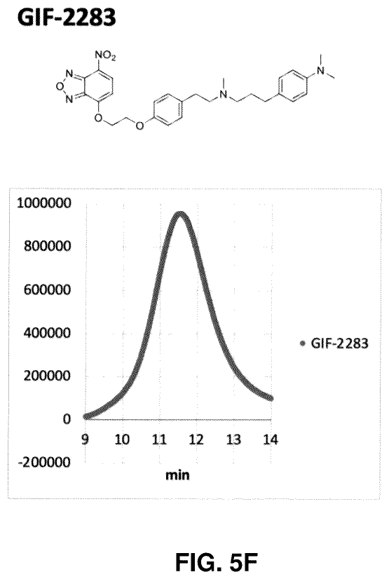

Experiments were performed in the same procedure as in Example 10 except that, instead of the pasteurized milk of Example 10, the lactic fermenting beverage (Yakult)-derived extracellular vesicles prepared in the procedure described in Example 12 was used. FIG. 5a to FIG. 5f illustrate the results. As is clear from the graphs illustrated in FIG. 5a to FIG. 5f, it was confirmed that the compounds synthesized in Examples 1 to 9 can be used as a staining agent for gel filtration of different types of extracellular vesicles. Note that, in the staining of the pasteurized milk of Example 10, the sensitivity was highest in the order of GIF-2281, GIF-2280, and GIF-2276. On the other hand, in the lactic acid bacteria of Example 14, the sensitivity was highest in the order of GIF-2277, GIF-2281, and GIF-2282. Therefore, it was confirmed that the compounds disclosed in the present application can be suitably selected in accordance with the type of extracellular vesicles to be stained. Selection of a compound with high sensitivity can reduce the amount of the compound to be used.

[Introduction of Extracellular Vesicles Labeled with Staining Agent into Cells and Tracking of the Same]

### Example 15: Milk-Derived Extracellular Vesicles

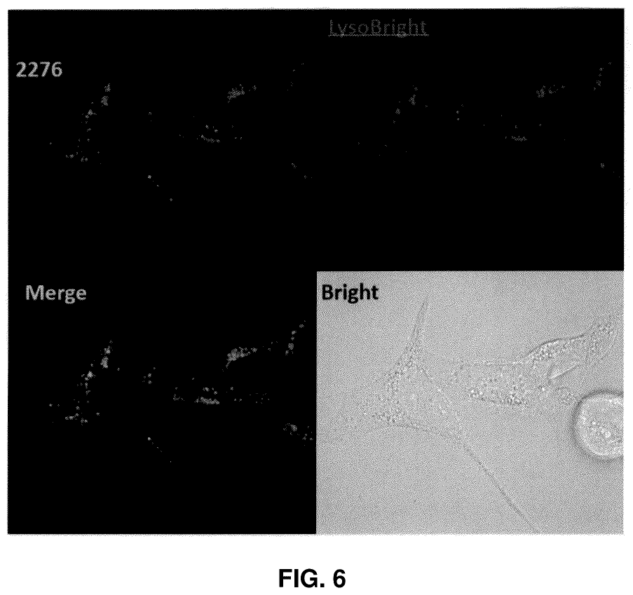

The milk-derived extracellular vesicles (exosomes) prepared in the procedure of Example 10 were stained with GIF-2276 at room temperature for 12 hours to label the extracellular vesicles. Unreacted GIF-2276 was removed by Microcon 300K (by Millipore). Lysosomes in HEK293 cells (ThermoFisher) were labeled with LysoBrite Red, and GIF-2276-labeled milk-derived extracellular vesicles were added thereto. After 3 hours, the mixture was observed by a microscope. FIG. 6 illustrates the result. The upper left “2276” in FIG. 6 is a fluorescent photograph of the extracellular vesicles labeled with GIF-2276. The upper right “LysoBright” in FIG. 6 is a fluorescent photograph of the lysosomes labeled with LysoBrite Red. The lower left in FIG. 6 is a photograph obtained by merging the fluorescent photograph of the extracellular vesicles labeled with GIF-2276 and the fluorescent photograph of the lysosomes labeled with LysoBrite Red. The lower right in FIG. 6 is a photograph of HEK293 cells captured in visible light. As is clear from the merged photograph in FIG. 6, most of signals from extracellular vesicles are localized in the lysosomes. From the above results, it was confirmed that the extracellular vesicle labeled with GIF-2276 can be tracked in the cells to which the same was introduced.

### Example 16: Lactic Acid Bacteria-Derived Extracellular Vesicles

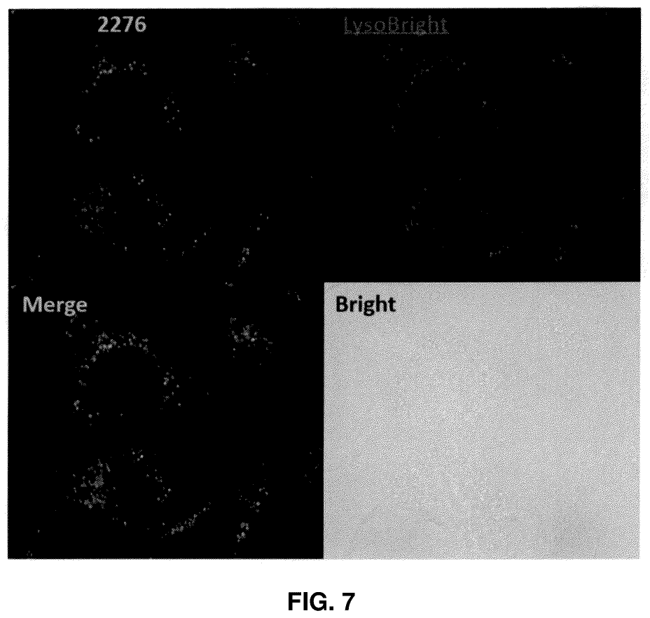

Experiments were performed in the same procedure as in Example 15 except that, instead of milk-derived extracellular vesicles, lactic acid bacteria (Yakult)-derived extracellular vesicles were used. FIG. 7 illustrates the result. As is clear from the merged photograph in FIG. 7, it was confirmed that, even when lactic acid bacteria (Yakult)-derived extracellular vesicles were used, the extracellular vesicle labeled with GIF-2276 can be tracked in the cells to which the same was introduced.

From the results of Examples 15 and 16, the compounds disclosed in the present application can label extracellular vesicles and enable tracking of the labeled extracellular vesicles in cells regardless of whether mammals or microorganisms are targeted. In the conventional method, tracking is made by relying on a particular marker, and such tracking is not possible in organism species where no marker is present. Although there is a tracking method in which a membrane is labeled in a nonspecific manner on the other hand, final tracking is difficult because the labeled compound will move to another membrane structure in the cell. In this regard, the compounds disclosed in the present application are excellent in that these compounds enable tracking of the fate of an extracellular vesicle without depending on organism species.

### Example 17 and Comparative Example 4: Comparison with Compound Disclosed in Patent Literature 1

### (1) Gel Filtration

The present applicant has disclosed GIF-2250 illustrated below in Patent Literature 1. GIF-2250 differs from the compounds of the present application in that the portion indicated by “→” is NH. First, the effect of this difference in structure provided on size separation using gel filtration was observed. Note that the synthesis procedure of GIF-2250 is disclosed in Patent Literature 1. The features disclosed in Patent Literature 1 are incorporated in the present specification by reference in its entirety.

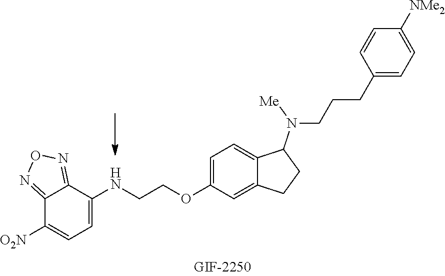 [SMILES: CN(C)c1ccc(CCCN(C)C2CCc3cc(OCCNc4ccc([N+](=O)[O-])c5nonc45)ccc32)cc1]

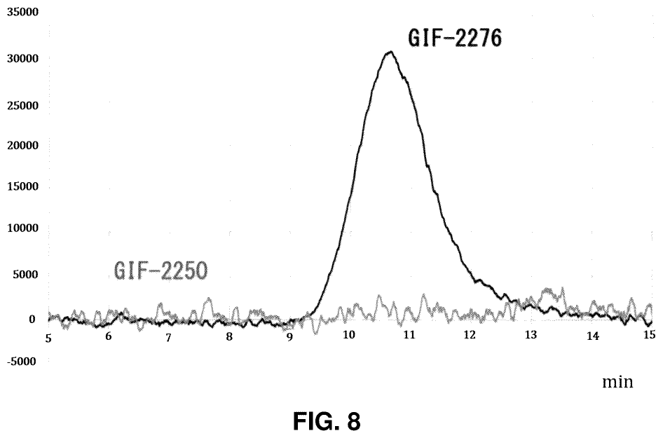

Experiments were performed in the same procedure as in Example 10 except that GIF-2276 and GIF-2250 (Comparative example 4), which is disclosed in Patent Literature 1, were used and the reaction time was 3 hours at room temperature. FIG. 8 illustrates a graph in which respective experimental results are overlapped. As is clear from FIG. 8, no peak of milk-derived extracellular vesicles was found in the extracellular vesicles stained by the compound of Patent Literature 1. In contrast, when GIF-2276 was used, a clear peak due to the size of milk-derived extracellular vesicles was found.

From the above results, it was confirmed that GIF-2250 disclosed in Patent Literature 1 can be used for use of staining of extracellular vesicles but not be used for size separation using liquid chromatography where a fluid pressure is applied. Further, the structural difference between each compound disclosed in the present application and GIF-2250 disclosed in Patent Literature 1 is in that the portion of “→” is “NH” or “O” as described above. Conceivable reasons why a fluid pressure caused the significantly different results to be obtained as illustrated in FIG. 8 are as follows:
(1) that GIF-2250 is only bonded with such force that causes easy separation from an extracellular vesicle due to a fluid pressure (for example, it is expected to be ionically bonded by weak electrostatic attraction); and
(2) that each compound disclosed in the present application, though the compound itself does not emit fluorescence, emits fluorescence in response to reacting with extracellular vesicles, which causes a change in the structure of the compound, and that the compound is bonded to an extracellular vesicle firmly enough to resist against a fluid pressure, and thus, in response to the compound coming into contact with an extracellular vesicle, a fluorescent group moiety is separated from the compound, and the fluorescent group moiety is covalently bonded to the component of the extracellular vesicle. Note that, from a view point of the structure of the fluorescent group, the separated fluorescent group is considered to be covalently bonded to the amino group of lysine on the surface of an extracellular vesicle.

### (2) Correlation Between Gel Filtration and Plate Reader

Patent Literature 1 discloses that the use of GIF-2250 enables quantification of extracellular vesicles by a plate reader. The correlation between detection of extracellular vesicles by a plate reader with the use of GIF-2250 and detection of extracellular vesicles by gel filtration with the use of GIF-2276 synthesized in Example 1 was examined.

Pasteurized milk (Takanashi) was heated at 60, 70, and 80° C. for 30 minutes and cooled back to room temperature. The extracellular vesicles were then prepared by the procedure described in Example 10, and the pasteurized milk was added thereto so that the final concentration of GIF-2276 synthesized in Example 1 became 1 μM to label the extracellular vesicles. The mixture was analyzed by a high-performance liquid chromatography gel filtration system to find the area of the peak. Extracellular vesicles prepared in the same manner reacted with 3 μM of GIF-2250 to find the fluorescence quantity by using a fluorescent plate reader. Respective values were plotted to find a correlation.

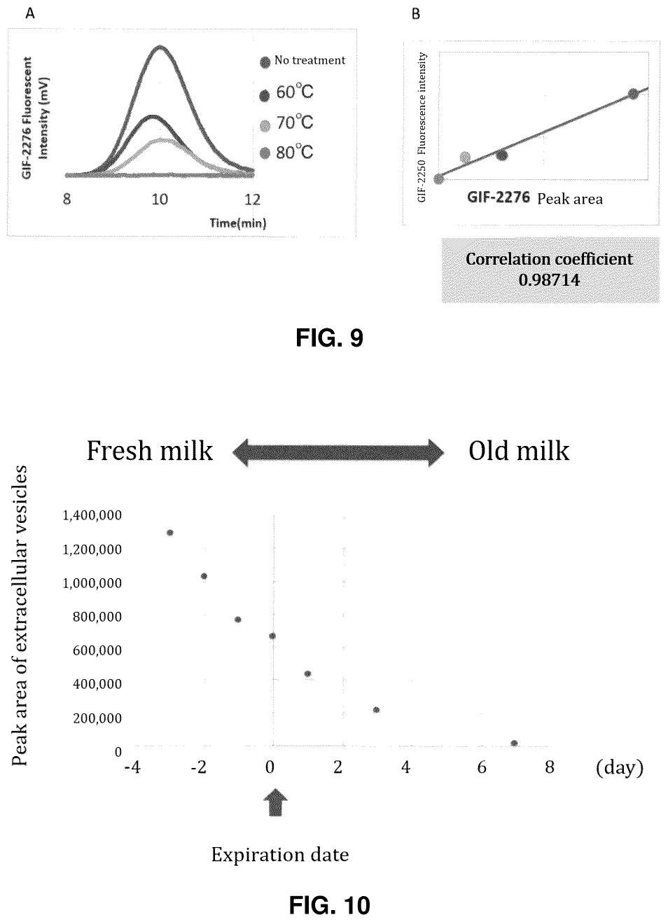

FIG. 9 indicates the result. A high correlation between the peak area due to gel filtration analysis of GIF-2276 and the fluorescence intensity of GIF-2250 was detected. While depiction is omitted, extracellular vesicles were successfully quantified with GIF-2276 even by a plate reader. Therefore, GIF-2276 can be used for quality control of extracellular vesicles in both methods with a plate reader and with gel filtration. In gel filtration analysis, it is possible to check the quality control of exosomes (whether or not the exosomes are destroyed) even from a difference in size. Therefore, the compound disclosed in the present application can be used for both the methods with a plate reader and with gel filtration and thus has an advantageous effect of enabling selection as to which method to use in accordance with the purpose of extracellular vesicle analysis.

### Example 18: Measurement of Degree of Freshness of Milk

GIF-2276 was used to analyze extracellular vesicles contained in milk with different degrees of freshness before the expiration date and after the expiration date where the expiration date was defined as day 0. The experiment was performed in the same procedure as in Example 10 except that milk with different degrees of freshness was used as the sample.

FIG. 10 indicates the result. As is clear from FIG. 10, it was confirmed that the quantity of measured extracellular vesicles changes in accordance with the degree of freshness of milk. Therefore, it was confirmed that the compound disclosed in the present application can be used for measuring the degree of freshness of foods.

### INDUSTRIAL APPLICABILITY

The present disclosure can be used for quantification and qualification of extracellular vesicles. Therefore, the present disclosure is useful in food industry, medical industry, or the like where quantification of extracellular vesicles is required.

1. A compound expressed by following Formula (1): 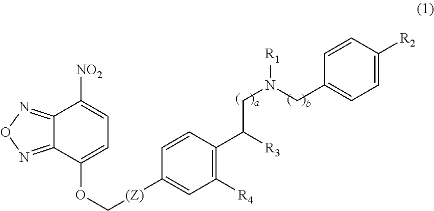
wherein in Formula (1), R₁ represents H, a C₁-C₆ alkyl group, a hydroxy group, an amino group, or a carboxy group, R₂ represents H, a C₁-C₁₈ alkyl group, a C₁-C₁₈ alkoxy group, NO₂ or N(CH₃)₂, and R₃ and R₄ each independently represent H or CH₃, where R₃ and R₄ may be bonded to each other to form a ring and, when R₃ and R₄ are bonded to each other to form a ring, R₃ and R₄ are CH₂, wherein “a” represents 0 or 1, and “b” represents 1, 2, or 3, and wherein Z represents a linker in which 0 to 12 chain molecules formed of elements selected from a group consisting of C, O, and N are bonded, and the chain molecules may include a cyclic structure or a branched chain.

2. The compound according to claim 1, wherein the compound expressed by Formula (1) is a compound expressed by following Formula (2): 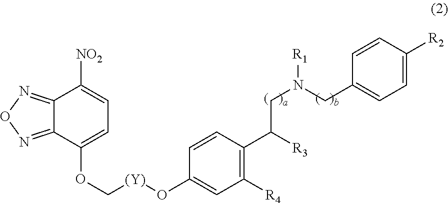
wherein in Formula (2), R₁ represents H or CH₃, R₂ represents H, CH₃, OCH₃, or N(CH₃)₂, R₃ and R₄ each independently represent H or CH₃, where R₃ and R₄ may be bonded to each other to form a ring and, when R₃ and R₄ are bonded to each other to form a ring, R₃ and R₄ are CH₂, wherein “a” represents 0 or 1, and “b” represents 1, 2, or 3, wherein Y represents a C₁-C₅ alkyl group, an ether chain represented by following Formula (2a), an ether chain including amido represented by following Formula (2b), or triazole represented by following Formula (2c), 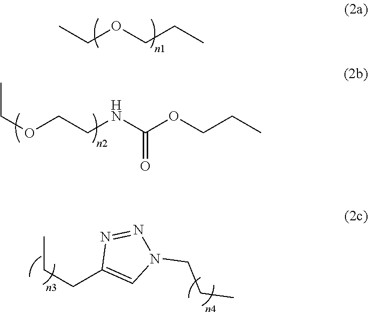
wherein in Formula (2a), n1 represents an integer of 1 to 3, wherein in Formula (2b), n2 represents an integer of 1 to 3, and wherein in Formula (2c), n3 represents an integer of 0 to 6, and n4 represents an integer of 0 to 3.

3. The compound according to claim 1, wherein the compound expressed by Formula (1) is a compound expressed by following Formula (3): 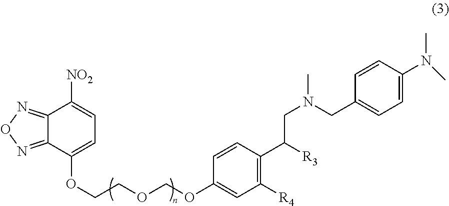
wherein in Formula (3), R₃ and R₄ each independently represent H or CH₃, where R₃ and R₄ may be bonded to each other to form a ring and, when R₃ and R₄ are bonded to each other to form a ring, R₃ and R₄ are CH₂, and wherein n represents an integer of 0 to 3.

4. An extracellular vesicle staining agent comprising the compound according to claim 1.

5. An extracellular vesicle staining agent comprising the compound according to claim 2.

6. An extracellular vesicle staining agent comprising the compound according to claim 3.

7. The extracellular vesicle staining agent according to claim 4 further comprising another fluorescent compound.

8. A method of fluorescent staining for extracellular vesicles, the method comprising:
a staining step of staining extracellular vesicles by using the compound according to claim 1; and
a detection step of detecting stained extracellular vesicles in a sample.

9. The method of fluorescent staining for extracellular vesicles according to claim 8 further comprising an evaluation step of evaluating the sample after the detection step.

10. The method of fluorescent staining for extracellular vesicles according to claim 9,
wherein the sample includes cosmetics, and
wherein the method measures the extracellular vesicle incorporated in cells to evaluate the cosmetics.

11. The method of fluorescent staining for extracellular vesicles according to claim 9,
wherein the sample includes pharmaceutical products, and
wherein the method measures the extracellular vesicles incorporated in cells to evaluate the pharmaceutical product.

12. The method of fluorescent staining for extracellular vesicles according to claim 9,
wherein the sample includes cells, and
wherein the method measures the extracellular vesicles in the cells to evaluate the state of the cells.

13. The method of fluorescent staining for extracellular vesicles according to claim 9,
wherein the sample includes foods or a beverages, and
wherein the method measures the extracellular vesicles in the foods or the beverages to evaluate the foods or the beverages.

14. The method of fluorescent staining for extracellular vesicles according to claim 9,
wherein the sample includes tissues collected from organisms, and
wherein the method measures the extracellular vesicles in the collected tissues to evaluate the tissues.

15. A method of fluorescent staining for extracellular vesicles, the method comprising:
a staining step of staining extracellular vesicles by using the compound according to claim 2; and
a detection step of detecting stained extracellular vesicles in a sample.

16. A method of fluorescent staining for extracellular vesicles, the method comprising:
a staining step of staining extracellular vesicles by using the compound according to claim 3; and
a detection step of detecting stained extracellular vesicles in a sample.

17. A method of fluorescent staining for extracellular vesicles, the method comprising:
a staining step of staining extracellular vesicles by using the extracellular vesicle staining agent according to claim 4; and
a detection step of detecting stained extracellular vesicles in a sample.

18. A method of fluorescent staining for extracellular vesicles, the method comprising:
a staining step of staining extracellular vesicles by using the extracellular vesicle staining agent according to claim 5; and
a detection step of detecting stained extracellular vesicles in a sample.

19. A method of fluorescent staining for extracellular vesicles, the method comprising:
a staining step of staining extracellular vesicles by using the extracellular vesicle staining agent according to claim 6; and
a detection step of detecting stained extracellular vesicles in a sample.
